(19) United States
(12) Patent Application Publication
(10) Pub. No.: US 2026/0287572 A1
(43) Pub. Date: Sep. 24, 2026

(54) FIELD TEST FOR DETERMINING EMULSIFIER CONCENTRATIONS IN DRILLING FLUIDS

(71) Applicant: Halliburton Energy Services, Inc., Houston, TX (US)

(72) Inventors: Sunita S. KADAM, Pune (IN); Harshada THAKAR, Pune (IN); Jay DEVILLE, Houston, TX (US); Jeffrey J. MILLER, Houston, TX (US); Preston Andrew MAY, Houston, TX (US)

(73) Assignee: Halliburton Energy Services, Inc., Houston, TX (US)

(21) Appl. No.: 19/087,898

(22) Filed: Mar. 24, 2025

(51) Int. Cl. G01N 33/28; G01N 1/38; G01N 21/27; G01N 21/29

(52) U.S. Cl. CPC G01N 33/2823; G01N 1/38; G01N 21/274; G01N 21/293; G01N 2001/386

(57) ABSTRACT

Oil-based treatment fluids, such as a drilling fluid, can include an emulsifier to help stabilize the phases of the fluid. A field test can be used to determine the initial or residual concentration of an emulsifier in the fluid. One or more reference samples with different concentrations of a reference emulsifier and a dye solution can be prepared. A volume of the fluid can be mixed with the dye solution and a supernatant, test sample can then be separated from solids in the fluid to be tested. Visual comparison of the color of the test sample against the reference sample or comparing the absorbance of the test and reference sample can be used to determine the concentration of emulsifier in the fluid. Comparison can be performed at a mud plant or wellsite. The absorbance of the test sample can be compared against a calibration curve generated from reference samples.

### TECHNICAL FIELD

A field test can be used to determine the concentration of an emulsifier in an oil-based drilling fluid. A drilling mud sample can be mixed with a dye solution to form an oil-soluble emulsifier and dye complex. Comparison of a test sample from the drilling mud against a reference sample can be used to determine the concentration of emulsifier in the drilling mud. Comparison can also include using pre-generated calibration curves to quantitatively determine the concentration of emulsifier in the fluid sample.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The features and advantages of various embodiments will be more readily appreciated when considered in conjunction with the accompanying figures. The figures are not to be construed as limiting any of the preferred embodiments.

FIG. 1 is a schematic showing the formation of an oil-soluble emulsifier and dye complex in an invert emulsion fluid according to certain embodiments.

FIGS. 2A-2E are photographs of several reference samples with a dye solution mixed with different concentrations of a reference emulsifier in a hydrocarbon liquid showing a change in color of the oil-soluble emulsifier and dye complexes with increasing concentrations of the emulsifier.

FIG. 3 shows a visual comparison of the color of a test sample against the colors of the reference samples of FIGS. 2A-2E to determine which reference sample the test sample color most closely matches.

FIG. 4 is a line graph of absorbance versus emulsifier concentration in oil, showing a linear relationship of absorbance to concentration.

FIG. 5 is a line graph of a calibration curve of absorbance versus emulsifier concentration for an emulsifier in a simulated drilling fluid sample, showing a linear relationship of absorbance to concentration.

FIG. 6 is a flow chart of using a calibration curve to determine the concentration of emulsifier in a drilling fluid.

FIG. 7 is a flow chart of forming a drilling fluid at a mud plant, testing the concentration of emulsifier, and transporting the drilling fluid to a wellsite.

FIG. 8 illustrates a system for introducing a drilling fluid into a subterranean formation to form a wellbore.

FIG. 9 is a flow chart of forming a wellbore with a drilling fluid and testing the concentration of residual emulsifier in the drilling fluid.

FIG. 10 is a bar graph showing actual emulsifier concentration results compared to a known concentration of emulsifier with varying emulsifier lot and barite type.

FIG. 11 is a bar graph showing actual emulsifier concentration results compared to a known concentration of emulsifier with varying water phase salinity and oil-to-water ratios.

FIG. 12 is a bar graph showing actual emulsifier concentration results compared to a known concentration of emulsifier with varying lime concentrations and solids contamination concentrations.

### DETAILED DESCRIPTION

Oil and gas hydrocarbons are naturally occurring in some subterranean formations. In the oil and gas industry, a subterranean formation containing oil and/or gas is referred to as a reservoir. A reservoir can be located under land or offshore. Reservoirs are typically located in the range of a few hundred feet (shallow reservoirs) to a few tens of thousands of feet (ultra-deep reservoirs). To produce oil or gas, a wellbore is drilled into a reservoir or adjacent to a reservoir. The oil, gas, or water produced from a reservoir is called a reservoir fluid.

As used herein, a “fluid” is a substance having a continuous phase that can flow and conform to the outline of its container when the substance is tested at a temperature of 71° F. (22° C.) and a pressure of one atmosphere “atm” (0.1 megapascals “MPa”). A fluid can be a liquid or gas. A homogenous fluid has only one phase; whereas a heterogeneous fluid has more than one distinct phase. A colloid is an example of a heterogeneous fluid. A heterogeneous fluid can be: a slurry, which includes a continuous liquid phase and undissolved solid particles as the dispersed or discontinuous phase; an emulsion, which includes a continuous liquid phase and at least one dispersed phase of immiscible liquid droplets; a foam, which includes a continuous liquid phase and a gas as the dispersed phase; or a mist, which includes a continuous gas phase and liquid droplets as the dispersed phase. As used herein, the term “base fluid” means the solvent of a solution or the continuous phase of a heterogeneous fluid and is often the liquid that is in the greatest percentage by volume of a treatment fluid. An emulsion has a continuous phase of water and immiscible liquid oil droplets as the dispersed or discontinuous phase or has a continuous phase of an oil and immiscible liquid water droplets as the dispersed or discontinuous phase. When naming an emulsion type, the first letter is the discontinuous phase. Accordingly, O/W is oil in water and is classified as an “emulsion”; whereas W/O is water in oil and is classified as an “invert emulsion”.

A well can include, without limitation, an oil, gas, or water production well, an injection well, or a geothermal well. As used herein, a “well” includes at least one wellbore. A wellbore can include vertical, inclined, and horizontal portions, and it can be straight, curved, or branched. As used herein, the term “wellbore” includes any cased, and any uncased, open-hole portion of the wellbore. A near-wellbore region is the subterranean material and rock of the subterranean formation surrounding the wellbore. As used herein, a “well” also includes the near-wellbore region. The near-wellbore region is generally considered to be the region within approximately 100 feet (30.5 meters) radially of the wellbore. As used herein, “into a subterranean formation” means and includes into any portion of the well, including into the wellbore, into the near-wellbore region via the wellbore, or into the subterranean formation via the wellbore.

Oil or gas operations can be performed using a treatment fluid. The term “treatment fluid” refers to the specific composition of the fluid as it is being introduced into a well. The word “treatment” in the term “treatment fluid” does not necessarily imply any particular action by the fluid. Examples of treatment fluids include, but are not limited to, drilling fluids, spacer fluids, workover fluids, cement compositions, completion fluids, and stimulation fluids.

During drilling operations, a wellbore is formed using a drill bit. A drill string can be used to aid the drill bit in drilling through a subterranean formation to form the wellbore. The drill string can include a drilling pipe. A treatment fluid adapted for this purpose is referred to as a “drilling fluid” or “drilling mud.” The wellbore defines a wellbore wall that is the exposed portion of the subterranean formation where the wellbore was formed. The drilling fluid may be circulated downwardly through the drilling pipe and back up the annulus between the wellbore wall and the outside of the drilling pipe. The drilling fluid performs various functions, such as cooling the drill bit, maintaining the targeted pressure in the well, and carrying drill cuttings upwardly through the annulus between the wellbore wall and the drilling pipe. Accordingly, a drilling fluid can possess desirable properties, such as viscosity or pumpability, to perform the various functions. A spacer fluid can be pumped through the wellbore to remove residual drilling fluid from the wellbore and prepare the wellbore for other oil and gas operations, such as a cementing operation, completion operation, or stimulation operation. To impart desirable properties to the treatment fluid, additives can be included in the fluid. One example of an additive is an emulsifier.

An emulsifier and a surfactant can have similar structures. While all emulsifiers can be classified as a surfactant, not all surfactants are emulsifiers. Emulsifiers generally stabilize a dispersion of an insoluble discontinuous phase (internal phase) in a continuous phase (external phase). Emulsifiers and surfactants are amphiphilic molecules comprising a hydrophobic portion and a hydrophilic portion. The hydrophilic portion can be charged. Emulsifiers and surfactants can lower the interfacial tension between two liquids or between a solid and a liquid. Emulsifiers and surfactants can also be used to change the wettability of the surface of solids of a formation. Wettability means the preference of a surface to be in contact with one liquid or gas rather than another. Accordingly, “oil-wet” means the preference of a surface to be in contact with an oil phase or gas phase rather than a water phase, and “water-wet” means the preference of a surface to be in contact with a water phase rather than an oil phase or gas phase. The wettability of a surface can be determined using a contact angle measurement. The contact angle is defined geometrically as the angle formed by a liquid at the three-phase boundary point where a liquid, gas, and solid intersect. A static contact angle can be measured using a goniometer. When water is used as the liquid, a static contact angle of less than 90° generally means the surface of the solid is water wet; and when oil is used as the liquid, a static contact angle of less than 90° generally means the surface of the solid is oil wet. Emulsifiers and surfactants can be used to change the wettability of the surface of the solids from being water-wet to being oil-wet or vice versa.

If an emulsifier is in a sufficient concentration in a solution, then the emulsifier molecules can form micelles. A “micelle” is an aggregate of emulsifier molecules dispersed in a solution. An emulsifier in an aqueous solution can form micelles with the hydrophilic portions in contact with the surrounding aqueous solvent, sequestering the hydrophobic portions in the micelle center. A micelle in a hydrocarbon solution forms with the hydrophobic portions in contact with the hydrocarbon solution, sequestering the hydrophilic portions in the center of the micelle. The emulsifier must be in a sufficient concentration to spontaneously form micelles, known as the critical micelle concentration (CMC). The critical micelle concentration is the concentration of emulsifier above which micelles are spontaneously formed.

The hydrophilic-lipophilic balance (“HLB”) of the emulsifier can be used to determine whether an emulsion forms as a water-in-oil or an oil-in-water emulsion. HLB is a measure of the degree to which an emulsifier is hydrophilic or lipophilic. One formula that can be used to calculate HLB is listed below as equation 1.

$$H L B = 20 * Mh / M \qquad Eq . 1$$

where Mh is the molecular mass of the hydrophilic portion of the emulsifier; and Mis the molecular mass of the emulsifier as a whole. The HLB can be used to define whether an emulsifier keeps a dispersed aqueous phase (e.g., water or oil) dispersed throughout the continuous phase and provides stability to the dispersed phase or whether the compound functions as a surfactant, such as changing the wettability of a surface. An emulsion or invert emulsion is considered to be stable when the continuous and discontinuous phases do not separate after the removal of agitation for at least an hour and solids do not separate out from the liquid phases. Generally, a compound can function as, and therefore can be categorized as, an emulsifier if the HLB of the compound is greater than 3.

Treatment fluids, such as drilling fluids, are prepared prior to beginning the oil and gas operation, such as a drilling operation. The drilling fluid can be prepared and formed at a central processing unit, such as a mud plant. As used herein, a “mud plant” is a facility that processes, stores, and manages drilling mud for oil and gas wells. A large volume (e.g., volumes in excess of 50 barrels or 2,100 gallons) of the drilling fluid or drilling mud can be prepared at the mud plant by combining the base fluid, liquid or gas dispersed phases, and additives. In general, volumes of around 500 barrels (21,000 gallons) or more are prepared in a dedicated mix tank which utilizes a large rotating agitator. Most plants are equipped with other circulating lines, a hopper for the addition of ingredients, and centrifugal pumping equipment that forces the fluid through multiple lines and back into the vessel to aid in thorough mixing of the two liquid phases with surfactants and other materials to homogenize them. In the same manner, existing volumes of fluid may be blended with additional ingredients to adjust the properties before subsequent drilling operations. The mud plant can serve a plurality of wellbores and drilling operations located within a geographic area, locality, field, or subterranean formation. After the drilling fluid is formed at the mud plant, the drilling fluid can be transported, for example by a pipeline or vehicle, to the particular wellsite where the drilling operation is to be performed.

A known concentration of additives, such as emulsifiers, are initially added to the fluid and has an “initial concentration.” As used herein, the “initial concentration” is the concentration of an additive that is added to the base fluid and other ingredients when the drilling fluid is formed (e.g., at the mud plant) and prior to use in a drilling operation. In cases where a drilling fluid is used at one wellsite and then transported to a different wellsite for use, the “initial concentration” also means the concentration of an additive prior to use at the different wellsite. It is desirable for the initial concentration of the additive to be within a range of a targeted concentration. In cases where the drilling fluid is transported to a different wellsite after use, additional amounts of the additive may need to be added before using the fluid at the different wellsite to bring the initial concentration within the range of the targeted concentration. The initial concentration can vary-depending on many factors such as the exact type of additive, desired properties of the drilling fluid, other ingredients in the drilling fluid, and the makeup and properties of the subterranean formation. Accordingly, the initial concentration of an emulsifier can be different if the fluid is transported to a different wellsite after use (e.g., the targeted concentration may be greater than or less than the targeted concentration when the fluid was formed at a mud plant).

Some oil or gas operations, for example drilling operations, generally take hours, days, or even weeks to complete. As used herein, any reference to a “drilling fluid” is meant to include other types of treatment fluids, including but not limited to, spacer fluids, cement compositions, completion fluids, workover fluids, and stimulation fluids. Moreover, as used herein, any reference to a “drilling operation” is meant to include other types of oil or gas operations, including but not limited to, cementing operations, workover operations, completion operations, and stimulation operations. During the drilling operation, the drilling fluid is circulated within the wellbore and back up to the surface where the fluid is cycled through solids removal equipment to remove drill cuttings among other things and recycled back into the wellbore. A drilling fluid can also be re-used at multiple job sites and recycled for months or even years. The concentration of the additives generally decreases during continued use of the drilling fluid. Accordingly, the “residual concentration” of the emulsifier in the drilling fluid is generally less than the initial concentration. As used herein, the term “residual concentration” means the concentration of the emulsifier in the drilling fluid after the drilling fluid has at least partially circulated in the wellbore. By way of example, if the initial, targeted concentration of emulsifier is 8 pounds per barrel (“ppb”) (22.8 kilograms per cubic meter “kg/m³”) and the drilling fluid is used for a period of time, then the residual concentration of the emulsifier may decrease to 5 ppb (14.3 kg/m³). More of the emulsifier can be added to the drilling fluid to bring the concentration back up to the targeted concentration, while the residual concentration can again become less than the targeted or initial concentration after being used again. Fluids heavily laden with solids such as lost-circulation particulates or finely ground drill solids (also known as low gravity solids) can further deplete the wetting capability of emulsifiers. “Heavily laden” refers to loadings of solids, such as lost-circulation materials, in a range of 20-80 pounds per barrel (or more), and drill solids or low-gravity solids in a range of about 50-150 pounds per barrel (or more). The latter type of solids are often very small particles, which accumulate in the drilling fluid over time. Lost-circulation materials, however, generally have a larger particle size, and may be added and removed in cycles during an oil or gas operation. It is therefore possible that the emulsifier coats the surfaces of the larger particles and can become depleted more rapidly during the addition and removal of the lost-circulation materials.

Depletion of emulsifiers can mean that there is no longer a sufficient concentration of the emulsifier to provide stability to the phases of an emulsion or invert emulsion or alter the wettability as needed. A fluid with too little emulsifier risks poor performance, for example “sagging” of other additives such as weighting agents and may lack adequate phase stability. Sagging of additives in a fluid can be determined using a sag factor, which is the bottom fluid density divided by the top fluid density plus the bottom fluid density-bottom fluid density/(top fluid density+bottom fluid density). A sag factor between 0.50 to 0.53 is generally considered acceptable to adequately keep additives from sagging and remain dispersed throughout the fluid. Sag factors greater than about 0.53 can indicate poor suspension properties. Sagging can also mean an increase in the density of the fluid of about 0.25 pounds per gallon or more. However, a fluid with excess emulsifier can be more costly because more of the additive is used than is needed, and the excess can also damage the subterranean formation and alter the reservoir wettability. The excess emulsifier can make the entire reservoir oil wet and give false readings on the reservoir content when formation evaluation activities are carried out. These may include indirect readings from sonic, acoustic, or gamma ray sensors, resistivity sensors, measurements of hydrocarbon saturation, or direct formation fluid sampling where liquids are selectively extracted from the formation zones of interest. By way of example, if the excess emulsifier changes the wettability of the reservoir to oil wet, then formation evaluation can become problematic. Reservoir characteristics, such as the residual oil saturation and the transition zone from oil to water saturation, may be very challenging to ascertain. Thus, false readings can make reservoir characterization and hydrocarbon production estimates very difficult when conducting exploratory or appraisal drilling.

In order to maintain a targeted concentration of emulsifiers in the drilling fluid prior to, during, or after use, a sample of the drilling fluid can be tested. Currently, samples must be sent off-site to a laboratory where testing such as mass spectrometry (MS), Fourier transform infrared (FTIR), nuclear magnetic resonance (NMR), or infrared (IR) spectroscopy can be performed because the size of testing equipment is too large for on-site testing. As used herein, the term “off-site” means at a location other than a wellsite where the fluid is used in an oil and gas operation or other than a mud plant where the fluid is formed. Such off-site testing can be very expensive due to the increased costs of having to ship the samples to the laboratory and a delay in the drilling operation while awaiting the results. There are also current procedures that are used to estimate the concentration of residual emulsifiers in the drilling fluid at the wellsite. The concentration of residual emulsifier in an oil-based drilling mud has traditionally been estimated at the wellsite from the appearance of the surface of the mud. Shiny surfaces indicated a sufficient concentration of emulsifier whereas “grainy” surfaces were an indication of an insufficient concentration. This approach is subjective and may not provide sufficient accuracy of the emulsifier concentration. Kjeldahl nitrogen measurements can also give an estimation of the total nitrogen in a drilling mud, though this procedure does not provide an indication of whether the emulsifier has been degraded or it is in a “healthy” state to provide the necessary functionality nor is the test selective for emulsifiers. Other approaches to on-site testing may only include qualitative means of estimating the emulsifier concentration. Thus, there is a need for being able to determine the concentration of emulsifier in a drilling fluid that does not require the sample to be sent offsite.

This disclosure allows the initial and/or residual concentration of emulsifier in a drilling fluid to be determined at the wellsite or other locations by comparing a test sample of the drilling fluid against one or more reference samples. A volume of the drilling mud can be mixed with a dye solution and can form an emulsifier and dye complex that is oil soluble when there is emulsifier present in the drilling fluid. Solids can be separated out and the supernatant, which is the test sample, can then be used to visually compare or compared using measured absorbance against the reference sample. The measured absorbance can be measured at a targeted wavelength using an instrument, such as a colorimeter, infrared (IR) spectrometer, or UV-VIS spectrometer, to then compare against a measured absorbance of the reference sample. A calibration curve of the reference samples can also be used to determine the initial or residual concentration of emulsifier in the drilling fluid. It is to be understood that any discussion regarding the reference sample(s), dye solution, test sample, etc. is intended to apply to all of the method embodiments without the need to repeat the discussion for each method embodiment.

It is to be understood that any reference to the test sample, reference sample, treatment fluid, drilling fluid, etc. applies to all the method embodiments throughout without the need to repeat the various embodiments for each method embodiment. The methods include comparing a test sample of a treatment fluid, such as a drilling fluid, against one or more reference samples. The methods include preparing the test sample. The methods of preparing the test sample include obtaining a volume of the drilling fluid. It is to be understood that as used herein, “volume” is used to represent a quantity, and it can be in units of mass or weight (e.g., pounds) or in units of physical volume (e.g., liters or cubic feet). As used herein, “the volume of the drilling fluid” is the whole drilling fluid that can include continuous and dispersed phases and soluble and/or insoluble additives. It is to be understood that unlike other techniques that centrifuge the drilling fluid to obtain top oil from the mud, the methods do not require, nor do they include separating out the top oil from the drilling mud volume to prepare the test sample. According to any of the embodiments, the methods do not include separating top from the volume of drilling fluid before adding a dye solution. Accordingly, a volume of the whole drilling fluid can be used in the test instead of top oil, which reduces the amount of time needed to determine the concentration of residual emulsifier. A volume sample of the whole drilling fluid can be collected and used to prepare the test sample. The method of obtaining the volume of the whole drilling fluid does not include obtaining a volume of the drilling fluid and then separating out the top oil from the rest of the sample to be used for analysis. As used herein, the term “top oil” means the oil phase from the drilling fluid that is separated from drilling solids and optionally the water phase if the drilling fluid includes an aqueous liquid. Top oil can be separated from the solids or the solids and water phase in the whole mud sample by centrifugation, for example. Using a volume of the whole drilling fluid can give more accurate results compared to just using the top oil because the dye solution can interact with all of the ingredients in the drilling mud, for example, by allowing the dye solution to interact with surfaces of the solids and other ingredients. Using the whole drilling mud instead of just the top oil can also provide faster results. The volume of the drilling fluid that is obtained can be added to a mixing container or directly into a centrifuge tube.

The drilling fluid can be formed at a processing facility, for example, a mud plant. The drilling fluid can be an oil-based mud (“OBM”). The drilling fluid can be a water in oil invert emulsion. The drilling fluid includes a base fluid. The base fluid can include dissolved materials or undissolved solids. The base fluid can include a hydrocarbon liquid oil as the continuous phase of the invert emulsion. The oil can be selected from the group consisting of a fractional distillate of crude oil; a fatty derivative of an acid, an ester, an ether, an alcohol, an amine, an amide, or an imide; a saturated hydrocarbon; an unsaturated hydrocarbon; a branched hydrocarbon; a cyclic hydrocarbon; and any combination thereof. Crude oil can be separated into fractional distillates based on the boiling point of the fractions in the crude oil. An example of a fractional distillate of crude oil is diesel oil. The saturated hydrocarbon can be an alkane or paraffin. The paraffin can be an isoalkane (isoparaffin), a linear alkane (paraffin), or a cyclic alkane (cycloparaffin). The unsaturated hydrocarbon can be an alkene, alkyne, or aromatic. The alkene can be an isoalkene, linear alkene, or cyclic alkene. The linear alkene can be a linear alpha olefin or an internal olefin.

The drilling fluid can include an aqueous phase including water. The water can be the dispersed or discontinuous phase of the invert emulsion. The water can be selected from the group consisting of freshwater, seawater, brine, brackish water, and any combination thereof in any proportion. The drilling fluid can include other ingredients, such as lime, weighting agents, and/or a water-soluble salt. Examples of the water-soluble salt include Group 1 halogens (e.g., sodium chloride, sodium bromide, potassium chloride, and potassium bromide), Group 2 halogens (e.g., calcium chloride, calcium bromide, and magnesium chloride), formate salts (e.g., sodium formate, potassium formate, and cesium formate), zinc bromide, and any combination thereof. The drilling fluid can have a water phase salinity (“WPS”). Water phase salinity is a factor showing the activity level of salt in the oil-based drilling fluid (invert emulsion fluid) and can indicate the concentration of dissolved salt in the water droplets within the fluid. The drilling fluid can have a water phase salinity in the range of about 100,000 to about 400,000 parts per million (ppm). Alternate dispersed phases can also be utilized in the drilling fluid where salt solutions are not practical for various reasons. In this case, the typical aqueous phase with salt can be replaced by glycols, polyglycols, aqueous solutions, and combinations thereof.

The drilling fluid can also have a targeted oil-to-water ratio (“OWR”). The treatment fluid can have an OWR in the range of 50:50 to 90:10. The oil-to-water ratio can be selected such that the oil (hydrocarbon liquid) of the base fluid is in the largest proportion and forms the continuous phase of the invert emulsion drilling fluid. In some cases, the drilling fluid can be formulated as an “all-oil” system without an aqueous phase. These fluids may take up minor amounts of water during use or might incorporate small amounts of an alternate dispersed phase to assist with stability or possibly to improve activation of other additives. The drilling fluid can also include other ingredients, including but not limited to, viscosifiers, an emulsifier activator, fluid loss additives, friction reducers, shale stabilizers, lime, lost-circulation materials, suspending agents, weighting agents, or pH adjusters.

A volume of a dye solution is added to the volume of the drilling fluid as one of the steps to prepare the test sample. The dye solution includes a dye. Examples of dyes that can be used include, but are not limited to, fluorescein, Brilliant-Yellow, Bromocresol-Green, sodium 1,2-naphthoquinone-4-sulfonate, Thymol-Blue, Bromophenol Blue, Clayton-Yellow, and combinations thereof. The dye can be in the form of a sodium salt of the dye to increase the dye's solubility in a solvent. The chemical structures of the dyes or sodium salts of the dyes are provided below.

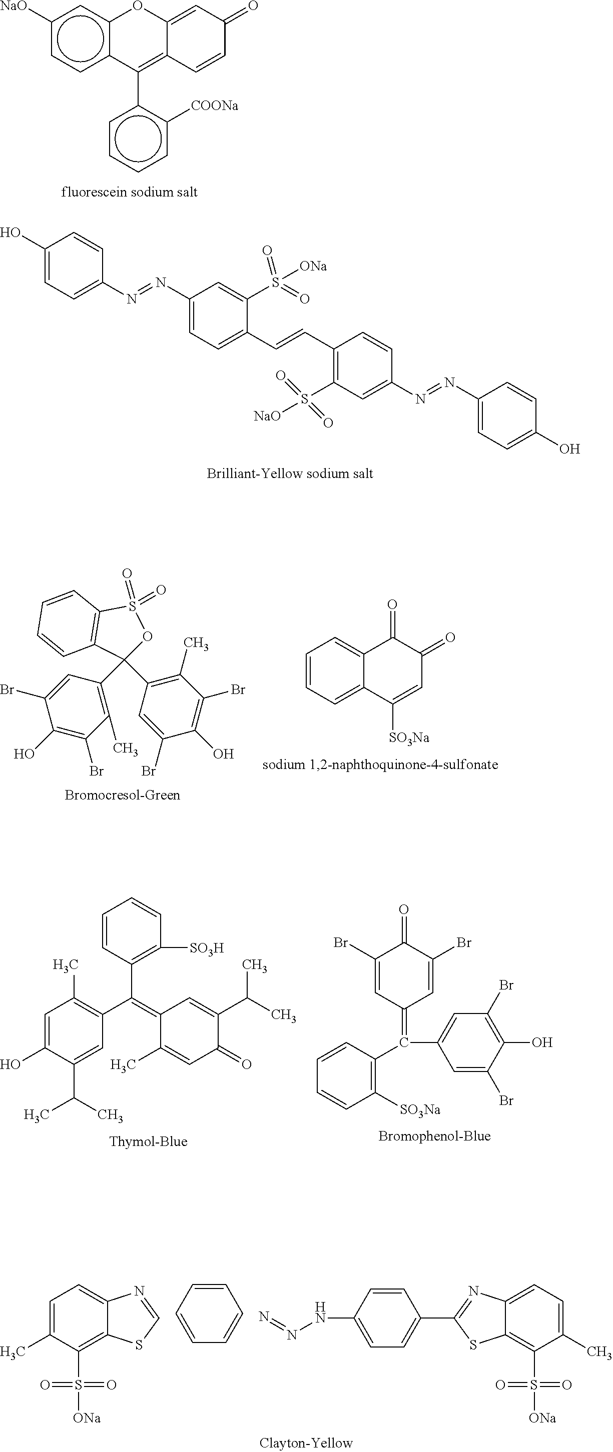

The dye solution can include a solvent for the dye. The solvent can be water or a polar organic solvent, such as an alcohol or a glycol. The alcohol can be selected from the group consisting of a primary alcohol (e.g., methanol, ethanol, propanol, butanol, etc.) or a secondary alcohol (e.g., 2-propanol or 2-butanol), or a tert-butanol. Other solvents can include ethylene glycol, propylene glycol, acetone, methyl ethyl ketone, dimethyl sulfoxide, dimethylformamide, or polyethylene glycol (PEG-200), and combinations thereof. The alcohol can be used as a solvent and for phase separation. That is, the dye can become highly soluble in the solvent when changing from water to organic solvents, which can change the color and/or absorption of the dye.

The dye solution can also include other ingredients, such as a pH adjuster, an acid, or an acidic buffer. The pH of the dye solution can be in a range of 1 to 5 or 3 to 4. Any pH adjuster, acid, or acidic buffer can be used to provide the targeted pH range. Examples of acids include but are not limited to acetic acid, citric acid, hydrochloric acid, formic acid, or lactic acid. The pH of the dye solution may be integral to the formation of the emulsifier and dye complex. According to any of the embodiments, the pH of the dye solution is adjusted-depending in part on the specific emulsifier and dye that is used-such that an oil-soluble, emulsifier and dye complex is formed.

The dye can be included in the dye solution at a concentration by weight percent (wt %) of the solvent. The concentration of the dye can be, for example, in a range of 0.005 to 5 wt %, or 0.005 to 1 wt. %, or 0.005 to 0.1 wt % of the solvent.

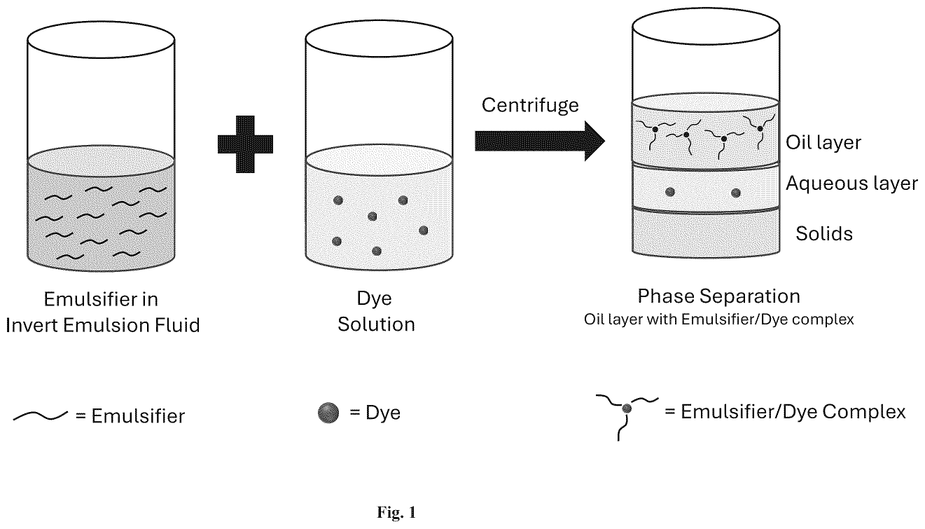

The dye solution can be added into a mixing container if used or into a centrifuge tube. An oil-soluble emulsifier and dye complex is formed when the emulsifier is still present in the drilling fluid, (i.e., the concentration of emulsifier in the drilling fluid is greater than 0 ppb). It is to be understood that if the drilling fluid has become completely depleted of the emulsifier, then an oil-soluble emulsifier and dye complex may not form. Being oil soluble means that the emulsifier and dye complex is formed in the oil phase of the drilling fluid. The solubility in the oil can include different factors, such as the pH of the dye solution, and the dye that is selected based on the type of emulsifier in the drilling fluid. The volume of drilling fluid to volume of the dye solution can vary and can be in a ratio of 3:1 to 1:1 or preferably 2:1 or 1:1. As can be seen in FIG. 1, the emulsifier from an invert emulsion fluid and the dye from the dye solution can form an emulsifier and dye complex in the oil phase. According to any of the embodiments, the emulsifier and dye complex is oil soluble. As used herein, the term “oil soluble” means at least 0.01 wt. % of the complex dissolves in 1 liter of an oil at a temperature of 71° F. (21.7° C.) and a pressure of 1 atmosphere.

The methods further include separating a supernatant from solids in the drilling fluid after the volume of the dye solution has been added to the volume of the drilling fluid. As used herein, the term “solids” includes insoluble particles, such as lost-circulation materials, when the drilling fluid was initially formed, for example to determine the initial concentration of the emulsifier in the drilling fluid; or they can include insoluble particles and drill cuttings or other solids that become part of the drilling fluid during the drilling operation, for example to determine the residual concentration of the emulsifier in the drilling fluid. The supernatant can include the oil phase of the drilling fluid and the oil-soluble emulsifier and dye complex. It is to be understood, that if the residual concentration of emulsifier in the drilling fluid is at or near 0 pounds per barrel, then the dye from the dye solution may not become oil soluble; and thus, according to this embodiment, the supernatant may not include the dye or the emulsifier and dye complex. The solids in the drilling fluid can include insoluble ingredients, for example, weighting agents, lost-circulation materials, and drill cuttings. The supernatant can be separated from the solids in the drilling fluid by allowing the mixture of the dye solution and drilling fluid to rest in the mixing container for a sufficient length of time (e.g., 30 minutes or more) so at least 50% of the solids settle to the bottom of the mixing container and the supernatant rises to the top of the mixing container. A demulsifier can be added to the container to help speed up the separation of the supernatant from the solids in the drilling fluid. The supernatant can also be separated from the solids via centrifugation. According to these embodiments, the drilling fluid and dye solution mixture is centrifuged after the dye solution has been added to the volume of drilling fluid. If the volume of the drilling fluid and the dye solution are added to a mixing container first, then the contents can then be transferred to a centrifuge tube. Centrifugation can be performed at a speed in the range of 500 to 4,500 revolutions per minute for example. Centrifugation can be performed for an amount of time, for example at least 1 minute, 5 minutes, 10 minutes, 20 minutes, or 30 minutes. The amount of time can be in a range of 1 minute to 120 minutes depending, in part, on the concentration of solids present in the drilling fluid and how long it will take for the solids to be forced to the bottom of the centrifuge tube. The amount of time can also be dependent on the speed of centrifugation. By way of example, a speed of 3,500 rpm may only need to be centrifuged for 5 minutes versus a longer time for a speed of 500 rpm. Centrifugation can allow the emulsifier and dye complex that is formed to rise to the surface in the centrifuge tube and force the solids to the bottom of the centrifuge tube.

The methods also include removing a volume of the supernatant after separation from the solids, wherein the volume of the supernatant is the test sample. As used herein, the “test sample” can be the entire volume of the supernatant or the test sample can be aliquoted from the volume of the supernatant. The volume of the supernatant can be removed after centrifuging or settling and can be withdrawn from the top of the mixing container or centrifuge tube. If formed, the emulsifier and dye complex is oil soluble. Accordingly, during the process of centrifugation or settling via gravity, the oil from the drilling fluid along with the emulsifier and dye complex (in the case where there is emulsifier in the drilling fluid) can rise to the top of the mixing container or centrifuge tube as a supernatant.

The test sample is compared against one or more reference samples. The methods can also include obtaining or preparing the one or more reference samples, wherein the reference samples include a known concentration of a reference emulsifier; the dye solution; and a hydrocarbon liquid. It is to be understood that as used herein, any discussion regarding “reference samples” includes a single reference sample (singular) and more than one reference sample (plural). The reference emulsifier can be the same emulsifier or a similar emulsifier as the emulsifier that has been or is to be included in the drilling fluid. A “similar emulsifier” can have a different HLB or different functional groups, for example, but can be of the same chemical family such as a polyamine. The reference emulsifier and emulsifier can be, for example, an amidoamine emulsifier. The amidoamine emulsifier can be for example a carboxylic acid terminated polyamine. The emulsifier can have a hydrophilic/lipophilic balance (HLB) greater than 3 or greater than 4. The emulsifier can have an HLB in the range of 3 to 6. There can also be more than one type of emulsifier in the reference samples and the drilling fluid to be tested. If there are two different types of emulsifiers in the drilling fluid to be tested, then the reference samples can include both types of emulsifiers. Alternatively, two separate sets of reference samples can be prepared, wherein the first set includes the first type of emulsifier, and the second set includes the second type of emulsifier. There can also be 3 or more types of different emulsifiers.

The reference samples also include a hydrocarbon liquid (e.g., oil), so an emulsifier and dye complex is formed in the oil. According to any of the embodiments, the hydrocarbon liquid in the reference samples is the same as the hydrocarbon liquid in the treatment fluid. According to other embodiments, the hydrocarbon liquid in the reference samples is similar to the hydrocarbon liquid in the drilling fluid. As used herein, a “similar” hydrocarbon liquid means chemically similar, for example, possessing the same functional groups with variations in the carbon chain lengths, or both hydrocarbon liquids belonging to the same class (e.g., a fatty derivative, cyclic hydrocarbon, etc.). The reference samples can also include water as a dispersed phase and can have a targeted oil-to-water ratio. The reference samples can include other ingredients in addition to the known concentration(s) of the reference emulsifier that simulate the drilling fluid, such as, but not excluding a water-soluble salt, lime, a weighting agent, a suspension agent, a viscosifier, a dispersant, a friction reducer, a fluid loss additive, a rheology modifier, lubricants, simulated drilled solids, and combinations thereof. In this manner, the reference samples can simulate the actual drilling fluid to be tested.

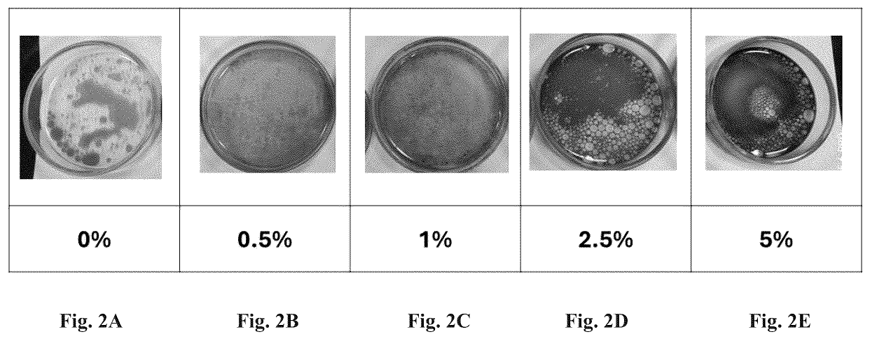

Turning to FIGS. 2A-2E, wherein 5 different reference samples were prepared with different concentrations of the reference emulsifier, the color of the emulsifier and dye complex can vary and can be dependent on the concentration of the reference emulsifier. By way of example and as shown in FIGS. 2A-2E when Bromophenol Blue was the dye used, as the concentration of the reference emulsifier increased from 0 wt. % to 5 wt. %, the color changed from a medium brown (FIG. 2A), to a brownish/green (FIGS. 2B and 2C), to greenish/blue (FIG. 2D), towards a darker blue (FIG. 2E).

The methods include comparing the test sample against the reference samples. The comparison against the reference sample can include visually comparing a color of the test sample against the color of the reference sample. “Color” has 3 components, which are the hue (i.e., the root color, for example, red, blue, yellow, or green); the value (i.e., how dark or light a color is); and the saturation (i.e., how gray or pure a color is). Thus, if the hue is blue, but one is darker than the other, then the colors are considered to be different from each other. Moreover, if one is pure blue and the other is a mixture of blue and green, then the colors are considered to be different from each other. A single reference sample can be prepared with a known concentration of the reference emulsifier. By way of example, if the targeted concentration of emulsifier is 2.5%, then a single reference sample can include the reference emulsifier at the known concentration of 2.5%. It is to be understood that the discussion of any “concentration” (i.e., initial, residual, or targeted) expressed as a percentage, includes a volume by volume (v/v) percent, a weight by volume (w/v) percent, or a weight by weight (w/w) percent. The color of the test sample can then be compared against the color of the single reference sample. If the colors are close to or approximate each other, then it can be determined that the concentration in the drilling fluid is or is close to the concentration of the reference emulsifier in the reference sample. As used herein, the phrases “are close to each other” or “approximates each other” means the colors have the same hue and a very similar value and saturation. By way of example and with reference to FIGS. 2A-2E, the color of FIG. 2B is close to or approximates the color of FIG. 2C; however, none of the colors of FIGS. 2C, 2D, and 2E approximate each other.

If the color of the test sample does not approximate the color of the single reference sample, then the methods can further include adding incremental amounts of the emulsifier to another volume of drilling fluid until the colors are close to each other. As used herein, “another volume of drilling fluid” can include obtaining a new volume of drilling fluid, or it can include removing more of the original volume of drilling fluid that was collected in the case where the entire volume of the drilling fluid that was initially collected was not used to prepare the test sample (i.e., there is still some drilling fluid left over from the initial collection). As used herein, the term “incremental amounts” can be any stepwise increase in concentration, for example in 0.25% increments, 0.5% increments, 1% increments, and so on. Accordingly, if the incremental amount is 0.25%, then 0.25% can be added and the color compared against the reference sample color; if the colors are not approximate, then an additional 0.25% can be added to the another volume of drilling fluid and compared again. This process can be repeated with the specific incremental amount until the colors approximate each other. The incremental amounts of the emulsifier that are added can be recorded, so it can be determined how much more emulsifier needs to be added to the drilling fluid to bring the concentration up to within range of the targeted concentration.

When the colors are not close to each other, it may be difficult to determine whether the concentration of emulsifier in the test sample is greater than or less than the concentration of reference emulsifier in the single reference sample. In this case, it may be helpful to compare the color of the test sample to the colors of multiple reference samples that have different known concentrations of the reference emulsifier—for example as shown in FIGS. 2A-2E. The methods can further include preparing more than one reference sample, wherein each of the reference samples have a different known concentration of the reference emulsifier, and wherein each of the reference samples has a different color from each other. By way of example and with reference to FIGS. 2A-2E, if the color of the test sample is brownish/green, but the targeted concentration is 5%, then it can be determined that the concentration of emulsifier in the drilling fluid is less than the targeted concentration. By way of another example, if the color of the test sample is greenish/blue, but the targeted concentration is 0.5%, then it can be determined that the concentration of emulsifier in the drilling fluid is greater than the targeted concentration.

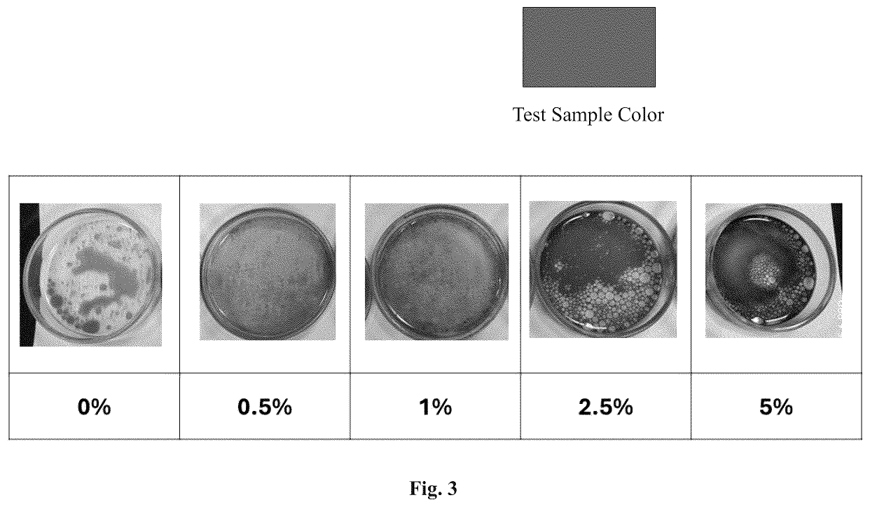

The methods can also include wherein the determination of the concentration of emulsifier in the drilling fluid comprises visually comparing a color of the test sample to the colors of the reference samples and determining which reference sample the color of the test sample most closely matches. By way of example and as shown in FIG. 3, the color of the test sample can be visually compared against the colors of the reference samples to find the reference sample whose color most closely matches the color of the test sample. In this example, the color of the test sample most closely matches or approximates the color of reference sample of FIG. 2D. If the test sample color included brown or yellow for example, then the color would most closely match the color of FIG. 2B or 2C.

The one or more reference samples can be prepared at the field-testing location where testing of the drilling fluid is to be conducted, for example at a mud plant or wellsite where the drilling operation is to be performed. According to this embodiment, a visual comparison can be made at the same location. The one or more reference samples can also be prepared offsite, for example in a laboratory or testing facility. The methods can further include obtaining a visual representation of the color(s) of the one or more reference samples. According to this embodiment, the person who prepares the reference sample can take photographs that visually represent and show the color of the reference samples that are labeled with the known concentration(s) of reference emulsifier. The photographs can be printed and taken to the field-testing location, or the photographs can be electronically sent to a person at the field-testing location, or the photographs can be copied onto a card that is then printed and taken to the field-testing location (similar to a color chart located on water chemical bottles that the color of a test strip can be matched against).

When preparing more than one reference sample, each one of the reference samples also includes a different concentration of the reference emulsifier from the other reference samples. The total number of reference samples that are prepared for comparison against the test sample can vary. Accordingly, it may be necessary to prepare a total of a minimum of 4 reference samples, or a minimum of 8 to 10 reference samples. The emulsifier concentration can be in a range from 0 to 20 pounds per barrel “ppb” (0 to 57.1 kilograms per cubic meter “kg/m³”) of the reference sample. According to any of the embodiments, the reference emulsifier concentration range in the reference samples is selected based on the anticipated or targeted concentration of emulsifier in the drilling fluid to be tested. By way of example, if the targeted concentration of emulsifier in the drilling fluid is 4 ppb (11.4 kg/m³), then the reference emulsifier concentration range in the reference samples can be 0 to 6 ppb (0 to 17.1 kg/m³). The unit increments in concentration between the reference samples can vary. The unit increments do not have to be the same for every point along the concentration range. By way of example, if the concentration range of the emulsifier is from 0 to 9 ppb, then there can be a total of 10 samples with a unit increment of 1 ppb. By way of another example, if the concentration range is from 0 to 5 ppb, then there can be a 0.25-unit increment from 0 to 2 ppb (9 reference samples) and then a 0.5-unit increment from 2 to 5 (6 reference samples) for a total of 15 reference samples.

The comparison of the test sample against the one or more reference samples can also be made by comparing the measured absorbance of the test sample to the measured absorbance of the one or more reference samples using an instrument, such as a colorimeter, UV-VIS spectrometer, or IR spectrometer. The methods can include measuring an absorbance of the reference sample using an instrument; and measuring an absorbance of the test sample using an instrument, wherein the comparison against the reference sample comprises comparing the absorbance of the test sample against the absorbance of the reference sample. The instrument used to measure the absorbance of the test sample can be the same or different type of instrument than was used to measure the absorbance of the reference sample. By way of example, a UV-VIS spectrometer can be used to measure the absorbance of the reference samples, and a colorimeter can be used to measure the absorbance of the test sample. It is to be understood that if the same type of instrument is used, it does not have to be the exact, same instrument, but rather is the same type of instrument (i.e., a UV-VIS spectrometer or a colorimeter). The comparison of absorbance values can be used to estimate an over/under concentration in the test sample. For example, if the absorbance of the test sample is greater than the absorbance of a single reference sample, then it can be determined that the concentration of emulsifier in the drilling fluid is greater than the targeted or initial concentration; and if the absorbance of the test sample is less than the absorbance of a single reference sample, then it can be determined that the concentration of emulsifier in the drilling fluid is less than the targeted or initial concentration.

The test sample can also be filtered before measuring the absorbance. The test sample can then be transferred into a cuvette for measuring the absorbance. According to any of the embodiments, the absorbance is measured at the field-testing location, for example at the wellsite or at a mud plant. The absorbance can be measured using a small testing device, such as a colorimeter. Using a colorimeter can be useful because a UV-VIS spectrometer may be too large to transport and keep at some wellsites. The absorbance can be measured at a targeted wavelength. Accordingly, the colorimeter that is used can be a single wavelength colorimeter that has the same wavelength as the targeted wavelength (e.g., 600 nm).

The methods can also include determining the targeted testing wavelength of the emulsifier and dye complex in the reference sample. The targeted testing wavelength can be the peak wavelength, which will be the wavelength with the highest absorbance peak, or a wavelength near the peak wavelength (e.g., +/−100 nanometers). At least one of the reference samples that includes the reference emulsifier at any concentration greater than 0, can be used to determine the targeted or peak wavelength of the emulsifier and dye complex. The targeted testing wavelength can vary based in part on the specific emulsifier and dye used to form the emulsifier and dye complex. According to the examples discussed below, the targeted testing wavelength was determined to be 600 nanometers (nm).

The instrument, such as a colorimeter (e.g., a photometer), a UV-VIS spectrometer, or an IR spectrometer can be used to measure the absorbance over a wavelength range to determine the targeted testing wavelength of the emulsifier and dye complex. The instrument measures the absorption of light to determine the concentration of the emulsifier in the sample, for example using the Beer-Lambert Law, which is the commonly used method to quantitatively determine the concentration of an absorbing species in solution. The instruments measure the intensity of light after passing through a sample (I), for example the reference samples or the test sample, and compares it to the intensity of the light before passing through the sample (I₀). The ratio of I/I₀ is called the transmittance and usually expressed as % T. The absorbance (A) is based on the transmittance according to equation 2 below.

$$A = - log ( \% T / 100 \% ) \qquad Eq . 2$$

The absorbance can also be determined using the Beet-Lambert Law according to equation 3 below.

$$A = {log}_{10} ( {I}_{0} / I ) \qquad Eq . 3$$

The concentration (c) of the emulsifier in the emulsifier and dye complex can then be determined using equation 4 below.

$$C = \frac{A}{ε L} \qquad Eq . 4$$

where A is the measured absorbance; ε is a constant known as the molar absorptivity or extinction coefficient that is a fundamental molecular property in a given solvent, at a particular temperature and pressure; and L is the path length through the sample. The instrument can provide the measured absorbance value via a display or printout, for example.

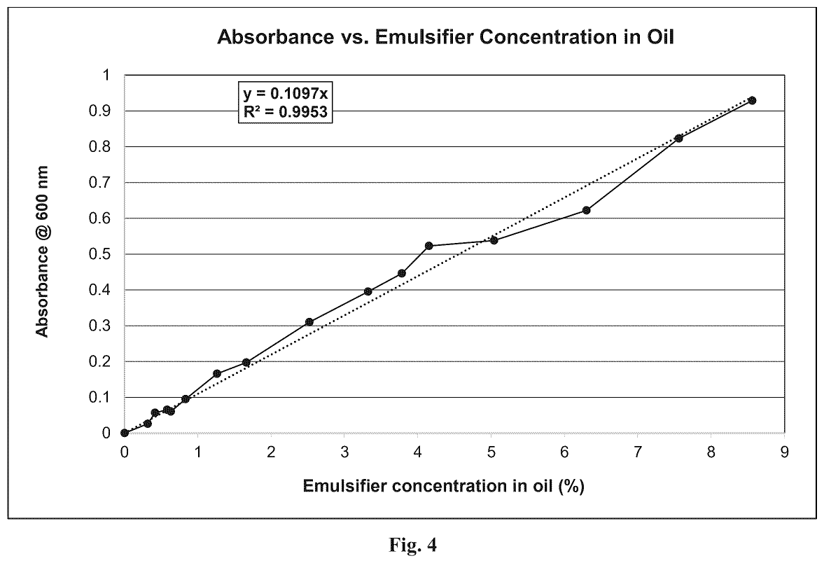

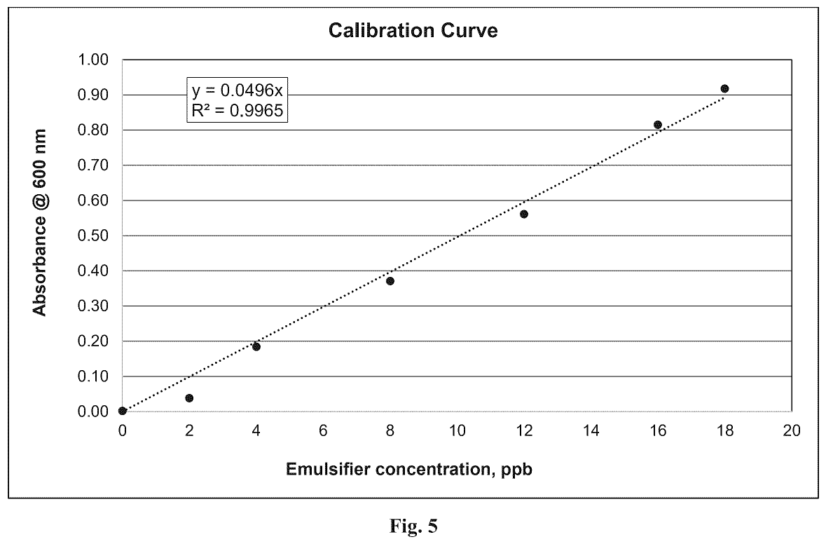

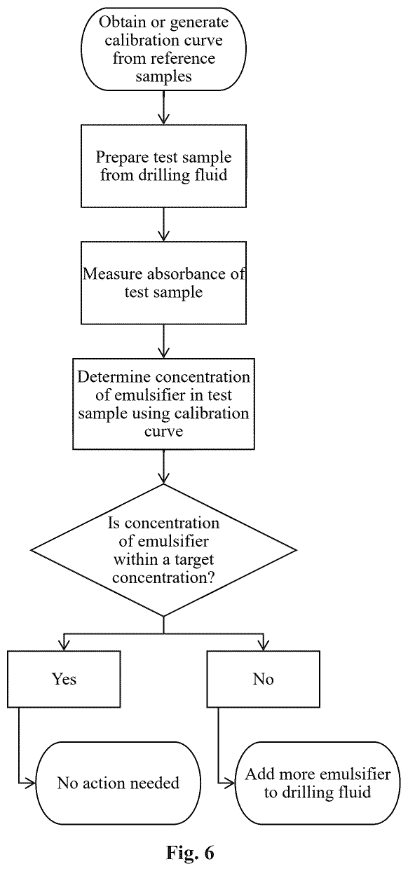

With reference to FIG. 6, the methods can also include obtaining or generating a calibration curve correlating absorbance to the reference emulsifier concentration from the reference samples. The absorbance of each of the reference samples can be measured at the targeted testing wavelength and plotted on a graph, for example as shown in FIGS. 4 and 5. According to any of the embodiments, the total number of reference samples that are prepared are selected such that a calibration curve can be generated over a range of reference emulsifier concentrations and to provide enhanced accuracy and reliability of the calibration curve. Statistical software can be used to fit the data to a linear regression of y=mx+b, where y is the absorbance at a testing wavelength, m is the slope, x is the reference emulsifier concentration, and b is the y-intercept. The plot should have absorbance values that are linear to the reference emulsifier concentration, and any area that is non-linear can indicate the limit of linearity. The coefficient of determination (R²) quantifies the goodness of fit, which is the square of the correlation coefficient between actual and predicted Y values. R² is generally a fraction where an R² of 1.0 is a perfect fit. The reference samples can be prepared, and the calibration curve generated off-site, for example in a laboratory or near a storage facility; or on-site at a mud plant or the wellsite where the drilling operation is to be performed. If the reference samples are prepared and the calibration curve is generated on-site, then a UV-VIS spectrometer may be too big to bring to the mud plant or wellsite, in which case a colorimeter can be used instead. A colorimeter is much smaller than a UV-VIS spectrometer and can even be a handheld device.

The test sample can be prepared from the drilling fluid after the step of obtaining or generating the calibration curve. The methods can include determining the concentration of the emulsifier in the drilling fluid by comparing the measured absorbance of the test sample to the calibration curve.

As discussed above, the targeted, initial concentration of emulsifier can become depleted through continued use of the drilling fluid as the emulsifier becomes bound to ingredients in the drilling fluid, lost into permeable areas of the subterranean formation, or bound to solid surfaces of the wall of the wellbore. Accordingly, while “the drilling fluid” comprises the base fluid, water, and one or more emulsifiers when initially formed at the targeted concentration, it is to be understood that at the time of testing, the residual concentration of emulsifier may be zero even though the drilling fluid included an initial concentration of emulsifier at some point before testing is performed. In other words, the emulsifier originally included in the drilling fluid may become completely depleted or only partially depleted during use or repeated use of the drilling fluid. It is to be understood that if the drilling fluid is tested at the time it is formed or before use at a different wellsite, then the initial concentration of emulsifier can be within a range (i.e., +/−10%) of the targeted concentration. However, if the testing is performed during or after use, then the residual concentration of emulsifier can be less than the initial concentration. The testing can be performed at multiple time intervals during use (e.g., every 1 hour, 2 hours, 4 hours, etc.) to provide real-time data on the residual concentration of emulsifier in the drilling fluid. Moreover, if after the testing is performed, and it is determined that the residual concentration of emulsifier is less than the range of the targeted concentration whereby additional emulsifier needs to be added, and the additional emulsifier is then added to the drilling fluid, then a re-test of the drilling fluid can be used to determine that the concentration falls within the range of the targeted concentration.

The methods can also include determining whether the concentration or residual concentration of the emulsifier in the drilling fluid is within the range of the targeted concentration. If the concentration of the emulsifier is within the targeted range, then additional emulsifier may not need to be added to the drilling fluid. However, if the concentration of the residual emulsifier is less than the targeted concentration, this can indicate that there is an insufficient amount of emulsifier in the drilling fluid. In this case, the methods can further include adding additional emulsifier to the drilling fluid, so the total concentration of emulsifier is withing the range (e.g., +/−10%) of the targeted concentration. The amount of emulsifier that needs to be added to the drilling fluid can be determined based on the specifics of the subterranean formation being drilled and other desirable properties of the drilling fluid. By way of example, if the targeted concentration of the emulsifier is 12 ppb (34.2 kg/m³) and the actual concentration of residual emulsifier is determined to be 6 ppb (17.1 kg/m³), then an additional 6 pounds (2.72 kg) of emulsifier can be added for each barrel of drilling fluid. The methods can further include re-testing the drilling fluid after adding additional emulsifier to the drilling fluid to determine if the amount of additional emulsifier brought the total concentration up to within the range of the targeted concentration. In this manner, it can be ensured that the total concentration is increased to be within the range of the targeted concentration after the additional emulsifier was added.

The ingredients in the drilling fluid, for example water-soluble salts, lime, or weighting agents, and the exact oil-to-water ratio can affect the accuracy of the absorbance of emulsifier in the test sample. By way of example, and as discussed below in the examples section, if a calibration curve was generated with reference samples containing 12 ppb of emulsifier and having a water phase salinity of 200,000, but the actual WPS of the drilling fluid test sample is 180,000, then the measured absorbance of the test sample may not accurately reflect an emulsifier concentration of 12 ppb. Accordingly, the reference samples that are used to generate the calibration curve can include ingredients other than the dye, solvent, pH adjuster, and varying concentrations of the reference emulsifier. To account for this possible discrepancy, several options can be used to enhance accuracy of the concentration determination.

For a first option, the actual ingredients and their concentrations can be determined from the actual drilling fluid that will be tested. By way of example, the actual drilling fluid to be tested can have a specific WPS, OWR, lime concentration, weighting agent concentration, lost-circulation materials, suspending agents, etc. The reference samples can then be made based on the specifics for that particular drilling fluid to simulate the drilling fluid except for the different concentrations of the reference emulsifier. It is to be understood that the sample of the drilling fluid that is tested may have already been used to drill a wellbore and may include drill cuttings and/or a reservoir fluid or other ingredients that become part of the drilling fluid during use; however, the simulated reference samples can, but do not need to, include drill cuttings or the other ingredients that became part of the drilling fluid during use and instead can be prepared based on the actual ingredients and concentrations of the drilling fluid before use. That is, representative drilling fluid samples can be prepared that have the identical ingredients and concentration of those ingredients except the different concentrations of the reference emulsifier, which can be called a base mud formulation. Then the different concentrations of emulsifier as discussed above to generate the calibration curve can be added to the base mud formulation. The calibration curve is then generated from reference samples that accurately simulate the specific drilling fluid formulation. According to this option, the reference samples can be made at a mud plant or the wellsite and the calibration curve generated on-site. A colorimeter may need to be used on-site, as discussed above. The reference samples can also be made offsite, for example, in a laboratory; however, this option may take longer to generate the calibration curve compared to doing so on-site.

For a second option, multiple calibration curves can be generated based on different sets of reference samples, wherein each set of reference samples has a different drilling fluid property. By way of example, one set of reference samples can be made having a WPS of 100,000, another set having a WPS of 125,000, another set having a WPS of 150,000, etc. According to this example, there can be 2, 4, 5, 10 or any other number of calibration curves that are generated based on varying WPS. Then, the calibration curve that is selected to compare the measured absorbance of the emulsifier in the drilling fluid test sample can be the calibration curve that was generated from the set of reference samples having the same or very similar WPS as that of the actual WPS of the drilling fluid. By way of example, if the WPS of the actual drilling fluid that is tested is 180,000, then the calibration curve generated from reference samples having a WPS of 180,000 can be used. Multiple calibration curves can be pre-generated with other properties such as OWR, lime concentration, etc. There can also be multiple calibration curves generated with different combinations of properties. By way of example, a calibration curve can be generated from reference samples having a WPS of 150,000, an OWR of 70:30, a lime concentration of 2 ppb, and a weighting agent concentration of 50 ppb; while another calibration curve can be generated from reference samples having a WPS of 175,000, an OWR of 75:25, a lime concentration of 3 ppb, and a weighting agent concentration of 45 ppb. The calibration curve that is selected can be the curve that most closely matches the actual properties of the drilling fluid to be tested.

For a third option, a correction factor can be used. The correction factor can be determined from control test samples, each having the same concentration of emulsifier and same dye solution. By way of example, if there are 3 samples: 1) a control reference sample (“ctl. ref. sample”) that was used to generate the calibration curve had a WPS of 250,000 and an emulsifier concentration (“conc.”) of 12 ppb with a specific measured absorbance; 2) a first control (“1^{st} ctl.”) test sample having a WPS of 180,000 gave an absorbance that when compared to the calibration curve yielded a concentration of 13.5 ppb; and 3) a second control (“2^{nd} ctl.”) test sample having a WPS of 280,000 gave an absorbance that when compared to the calibration curve yielded a concentration of 10.9 ppb, then it can be known that the measured concentration from the control test samples against the control reference sample was not accurate-because the properties (the WPS in this example) of the first and second control test samples were different from the control reference sample-rather it read higher or lower than actual (13.5 ppb in the high case or 10.9 ppb in the low case versus the actual of 12 ppb). In this instance, a correction factor can be used on any test fluids having a different fluid property (e.g., WPS, OWR, lime concentration, etc.) than the control test samples. Then, if the drilling fluid to be tested has an actual WPS of 175,000, it is known the concentration will be inaccurately high, so the correction factor can be subtracted from the concentration of residual emulsifier determined from the calibration curve; and it can be added to any drilling fluid that has a WPS greater than 250,000. The correction factor may not be 100% accurate, but it can give a more accurate concentration than without using the correction factor. However, the more control test samples that are included in the average can provide a more accurate correction factor. If there is any doubt whether the concentration determined at the wellsite for the drilling fluid that is tested is accurate—even if taking into account a pre-determined correction factor—then a new calibration curve can be generated at the wellsite where the reference samples include the specific fluid property, for example as described in the first option above. A correction factor can be calculated by taking the average of the numbers calculated using equation 5 below as an example for WPS.

$$\frac{( conc . ctl . ref . sample ( 12 ) * 1 st or 2 nd ctl . test WPS ( 18 , 000 or 280 , 000 )}{( 1 st or 2 nd ctl . test conc . ( 13.5 or 10.9 ) * ctl . ref . sample WPS ( 250 , 000 )} = average of 0.93651 \qquad Eq . 5$$

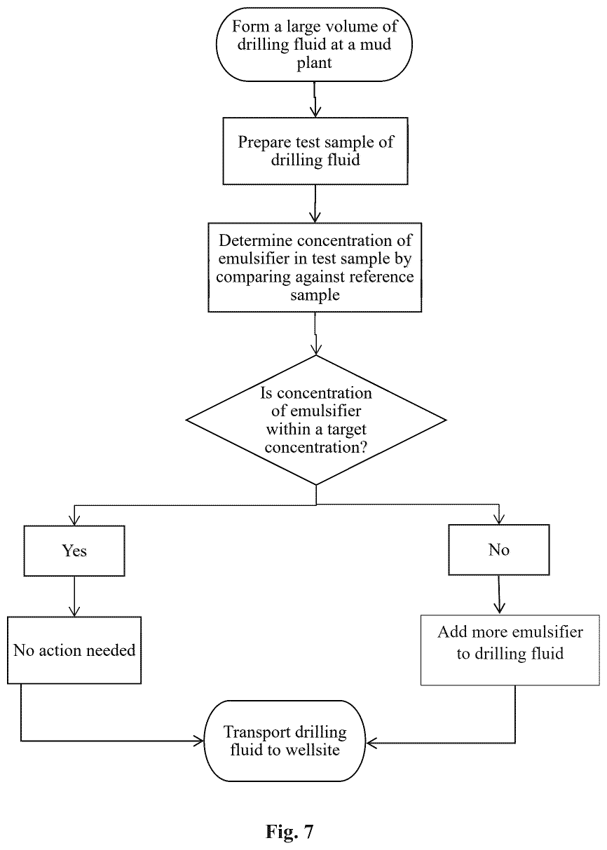

Turning to FIG. 7, the methods can include forming a volume of drilling fluid at a mud plant. The volume of drilling fluid that is formed, in other words, prepared, can be a large volume of drilling fluid (i.e., greater than 50 barrels or 2,100 gallons). The mud plant can be near the wellsite. The mud plant can provide the drilling fluid to multiple wellsites. The test sample can then be prepared after formation of the drilling fluid. The concentration of emulsifier in the drilling fluid can then be determined by comparison against the one or more reference samples. The concentration of emulsifier that is determined can be the initial concentration before the drilling fluid is used in any drilling operation. If the concentration that is determined is within a range of or greater than the target concentration, then no additional action is needed. However, as discussed above, if the concentration is less than the minimum of the range, then additional amounts of the emulsifier can be added to the drilling fluid. The methods can further include transporting the drilling fluid to the wellsite where the drilling operation is to be performed. Transportation can occur after the concentration of emulsifier in the drilling fluid is within the range of the target concentration, or the drilling fluid can first be transported to the wellsite and then additional emulsifier added to the drilling fluid at the wellsite.

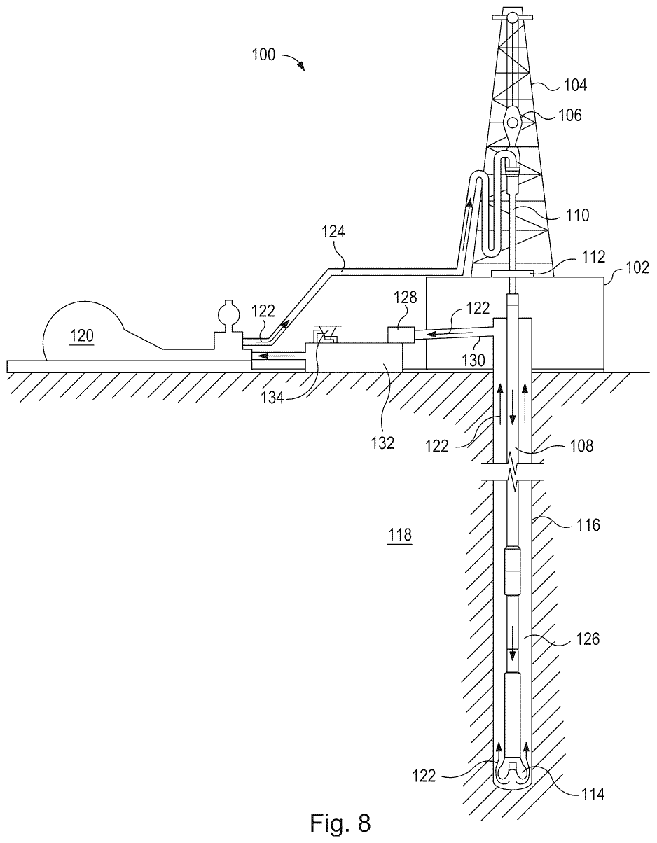

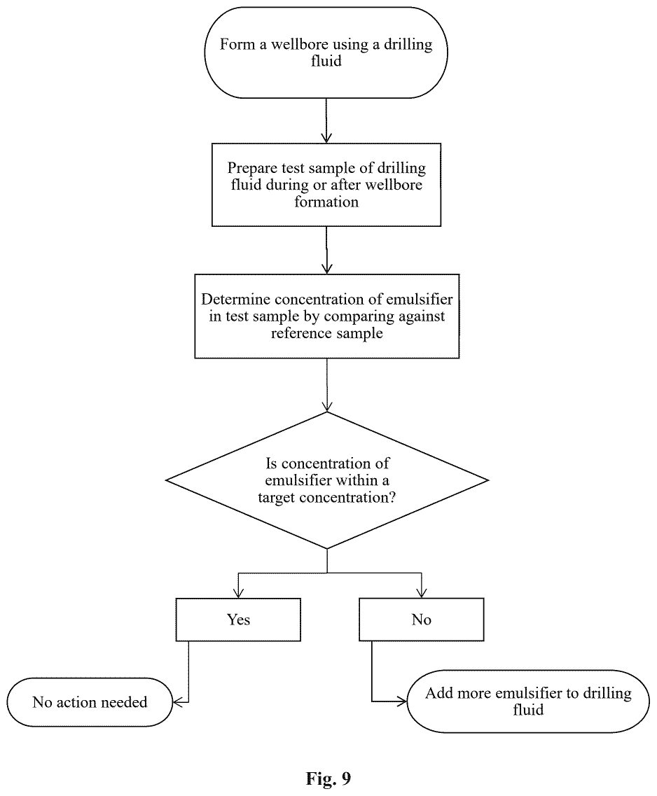

Referring now to FIGS. 8 and 9, the methods can also include introducing the drilling fluid into a subterranean formation for forming a wellbore, wherein the drilling fluid has an initial concentration of the emulsifier. The methods according to this embodiment can also include determining the initial concentration by comparing the test sample against reference samples either at the mud plant as discussed above or at the wellsite. A final test sample can be prepared at the conclusion of the drilling operation and compared against the reference samples, which may be useful to determine a “final concentration” before the drilling fluid is transported back to the mud plant, stored, or sent to a different wellsite for use.

The methods according to this embodiment include comparing the test sample against the reference sample(s) to determine the residual concentration of the emulsifier in the drilling fluid as it may become depleted during drilling of the wellbore. As discussed above, if the residual concentration of the emulsifier is less than the range of the targeted concentration, then additional emulsifier can be added to the drilling fluid, for example on-the-fly. Multiple testing can be performed as the drilling fluid is used to form the wellbore. For example, a test sample can be prepared every 2 hours, every 4 hours, or any other time intervals as needed, to determine the residual concentration of the emulsifier during use.

The drilling fluids and other treatment fluids disclosed herein can directly or indirectly affect one or more components or pieces of equipment associated with the preparation, delivery, recapture, recycling, reuse, and/or disposal of the disclosed fluids. For example, and with reference to FIG. 8, the disclosed fluids can directly or indirectly affect one or more components or pieces of equipment associated with an exemplary wellbore drilling assembly 100. It should be noted that while FIG. 8 generally depicts a land-based drilling assembly, those skilled in the art will readily recognize that the principles described herein are equally applicable to subsea drilling operations that employ floating or sea-based platforms and rigs, without departing from the scope of the disclosure. It should also be noted that while FIG. 8 and the discussion of the figure is for a drilling operation and fluid, other types of treatment fluids, such as spacer fluids, workover fluids, and stimulation fluids can be used with the disclosed equipment.

As illustrated, the drilling assembly 100 can include a drilling platform 102 that supports a derrick 104 having a traveling block 106 for raising and lowering a drill string 108. The drill string 108 can include, but is not limited to, drill pipe and coiled tubing, as generally known to those skilled in the art. A kelly 110 supports the drill string 108 as it is lowered through a rotary table 112. A drill bit 114 is attached to the distal end of the drill string 108 and is driven either by a downhole motor and/or via rotation of the drill string 108 from the well surface. As the bit 114 rotates, it creates a borehole 116 that penetrates various subterranean formations 118.

A pump 120 (e.g., a mud pump) circulates drilling fluid 122 through a feed pipe 124 and to the kelly 110, which conveys the drilling fluid 122 downhole through the interior of the drill string 108 and through one or more orifices in the drill bit 114. The drilling fluid 122 is then circulated back to the surface via an annulus 126 defined between the drill string 108 and the walls of the borehole 116. At the surface, the recirculated or spent drilling fluid 122 exits the annulus 126 and can be conveyed to one or more fluid processing unit(s) 128 via an interconnecting flow line 130. After passing through the fluid processing unit(s) 128, a “cleaned” drilling fluid 122 is deposited into a nearby retention pit 132 (i.e., a mud pit). A mud pit is an open pit dug into the ground or a tank or other receptacle for holding drilling fluid before it is circulated back into the feed pipe 124. While illustrated as being arranged at the outlet of the wellbore 116 via the annulus 126, those skilled in the art will readily appreciate that the fluid processing unit(s) 128 can be arranged at any other location in the drilling assembly 100 to facilitate its proper function, without departing from the scope of the disclosure.

One or more additional fluids can be added to the drilling fluid 122 via a mixing hopper 134 communicably coupled to or otherwise in fluid communication with the retention pit 132. The mixing hopper 134 can include, but is not limited to, mixers and related mixing equipment known to those skilled in the art. In other embodiments, however, the additional fluids can be added to the drilling fluid 122 at any other location in the drilling assembly 100. In at least one embodiment, for example, there could be more than one retention pit 132, such as multiple retention pits 132 in series. Moreover, the retention pit 132 can be representative of one or more fluid storage facilities and/or units where the disclosed fluids can be stored, reconditioned, and/or regulated until added to the drilling fluid 122.

As mentioned above, the disclosed fluids can directly or indirectly affect the components and equipment of the drilling assembly 100. For example, the disclosed fluids can directly or indirectly affect the fluid processing unit(s) 128 which can include, but is not limited to, one or more of a shaker (e.g., shale shaker), a centrifuge, a hydrocyclone, a separator (including magnetic and electrical separators), a desilter, a desander, a separator, a filter (e.g., diatomaceous earth filters), a heat exchanger, or any fluid reclamation equipment. The fluid processing unit(s) 128 can further include one or more sensors, gauges, pumps, compressors, and the like used to store, monitor, regulate, and/or recondition the disclosed fluids.

The disclosed fluids can directly or indirectly affect the pump 120, which representatively includes any conduits, pipelines, trucks, tubulars, and/or pipes used to fluidically convey the fluids downhole, any pumps, compressors, or motors (e.g., topside or downhole) used to drive the fluids into motion, any valves or related joints used to regulate the pressure or flow rate of the fluids, and any sensors (i.e., pressure, temperature, flow rate, etc.), gauges, and/or combinations thereof, and the like. The disclosed fluids can also directly or indirectly affect the mixing hopper 134 and the retention pit 132 and their assorted variations.

The disclosed fluids can also directly or indirectly affect the various downhole equipment and tools that can come into contact with the fluids such as, but not limited to, the drill string 108, any floats, drill collars, mud motors, downhole motors and/or pumps associated with the drill string 108, and any MWD/LWD tools and related telemetry equipment, sensors or distributed sensors associated with the drill string 108. The disclosed fluids can also directly or indirectly affect any downhole heat exchangers, valves and corresponding actuation devices, tool seals, packers and other wellbore isolation devices or components, and the like associated with the wellbore 116. The disclosed fluids can also directly or indirectly affect the drill bit 114, which can include, but is not limited to, roller cone bits, PDC bits, natural diamond bits, any hole openers, reamers, coring bits, etc.

While not specifically illustrated herein, the disclosed fluids can also directly or indirectly affect any transport or delivery equipment used to convey the fluids to the drilling assembly 100 such as, for example, any transport vessels, conduits, pipelines, trucks, tubulars, and/or pipes used to fluidically move the fluids from one location to another, any pumps, compressors, or motors used to drive the fluids into motion, any valves or related joints used to regulate the pressure or flow rate of the fluids, and any sensors (i.e., pressure and temperature), gauges, and/or combinations thereof, and the like.

The one or more test samples can be prepared by collecting a sample volume of the drilling fluid from a retention pit, for example, during and/or after use of the drilling fluid. The methods for a drilling operation can further include performing subterranean formation valuation prior to, during, or after the drilling operation. The methods can further include performing formation valuation, for example using a well logging tool, during the well-drilling operation, called measuring while drilling (MWD). During the well-drilling process, or shortly thereafter, instruments are passed through the wellbore to collect information about the formations through which the wellbore passes. The information is traditionally collected in “log” form, i.e., a table or chart of measured data values as a function of instrument position. The targeted concentration of emulsifier in the drilling fluid can change during the drilling operation as the valuation information is collected. Accordingly, the targeted concentration can increase or decrease with changes in the formation or between different formations through which the wellbore penetrates. For example, some formations consist of weak dispersive clays and shales, which can be broken up into fine solid particles by the drilling process. This results in a large amount of surface area that is added to the fluid system, which may be water-wet. According to this example, the targeted concentration of the emulsifier would need to be increased so the emulsifier can coat the increased surface area and/or change the wettability to oil wet. The amount of emulsifier that is added to the drilling fluid in response to the changes in the targeted concentration can be fine-tuned in a more effective manner without adding too much emulsifier and ensuring a minimum concentration is present, so the emulsifier functions as intended and needed. According to these embodiments, it is advantageous to prepare a new test sample whenever the targeted concentration changes in response to the valuation of the formation.

An embodiment of the present disclosure is a method comprising: 1) preparing a test sample comprising the steps of: (i) obtaining a volume of a treatment fluid; (ii) adding a volume of a dye solution to the volume of treatment fluid, wherein an oil-soluble emulsifier and dye complex is formed when the concentration of an emulsifier in the treatment fluid is greater than 0 pounds per barrel; (iii) separating a supernatant from solids in the treatment fluid, wherein the supernatant comprises the oil-soluble emulsifier and dye complex, if formed; and (iv) removing a volume of the supernatant, wherein the supernatant is the test sample; and 2) determining a concentration of the emulsifier in the treatment fluid by comparing the test sample against a reference sample, wherein the reference sample comprises: (i) a known concentration of a reference emulsifier; (ii) the dye solution; and (iii) a hydrocarbon liquid. Optionally, the concentration of the emulsifier that is determined is an initial concentration of emulsifier prior to using the treatment fluid in an oil and gas operation. Optionally, the concentration of the emulsifier that is determined is a residual concentration of emulsifier during or after use of the treatment fluid in an oil or gas operation. Optionally, the comparison against the reference sample comprises visually comparing a color of the test sample against a color of the reference sample. Optionally, the method further comprises adding incremental amounts of the emulsifier to another volume of the treatment fluid until the color of the supernatant approximates the color of the reference sample. Optionally, the method further comprises: preparing more than one reference sample, wherein each of the reference samples have a different known concentration of the reference emulsifier, and wherein each of the reference samples has a different color from each other; and wherein the determination of the concentration of emulsifier in the treatment fluid comprises visually comparing a color of the test sample to the colors of the reference samples and determining which reference sample the color of the test sample most closely matches. Optionally, the method further comprises: measuring an absorbance of the reference sample using an instrument; and measuring an absorbance of the test sample using an instrument, wherein the comparison against the reference sample comprises comparing the absorbance of the test sample against the absorbance of the reference sample. Optionally, the dye solution comprises a dye selected from the group consisting of Brilliant-Yellow, Bromocresol-Green, sodium 1,2-naphthoquinone-4-sulfonate, Thymol-Blue, Bromophenol Blue, Clayton-Yellow, and combinations thereof. Optionally, the dye solution further comprises a solvent for the dye. Optionally, the concentration of the dye is in a range of 0.005 to 5 wt % of the solvent. Optionally, the dye solution has a pH in a range of 1 to 5. Optionally, the reference emulsifier and the emulsifier comprise an amidoamine emulsifier. Optionally, the reference sample further comprises an ingredient selected from the group consisting of water, a water-soluble salt, lime, a weighting agent, a suspension agent, a viscosifier, a dispersant, a friction reducer, a fluid loss additive, a rheology modifier, lubricants, simulated drilled solids, and combinations thereof. Optionally, the ratio of the volume of treatment fluid to the volume of the dye solution is in a range of 3:1 to 1:1. Optionally, the supernatant is separated from the solids via centrifugation, and wherein the centrifugation is performed at a speed in a range of 500 to 4,500 revolutions per minute. Optionally, the centrifugation is performed for a period of time in a range of 1 minute to 120 minutes. Optionally, the method further comprises adding additional emulsifier to the treatment fluid after determination of the concentration of emulsifier in the test sample to bring the total concentration of emulsifier in the treatment fluid to within a range of a target concentration.

Another embodiment of the present disclosure is a method comprising: obtaining or generating a calibration curve correlating absorbance to an emulsifier concentration from reference samples, wherein the reference samples comprise: different concentrations of a reference emulsifier; a dye solution; and a hydrocarbon liquid; preparing a test sample comprising the steps of: obtaining a volume of a treatment fluid; adding a volume of the dye solution to the volume of the treatment fluid, wherein an oil-soluble emulsifier and dye complex is formed when the concentration of an emulsifier in the treatment fluid is greater than 0 pounds per barrel; separating a supernatant from solids in the treatment fluid, wherein the supernatant comprises the oil-soluble emulsifier and dye complex, if formed; and removing a volume of the separated supernatant, wherein the volume of the supernatant is the test sample; measuring an absorbance of the test sample using an instrument; and determining a concentration of the emulsifier in the test sample by comparing the measured absorbance to the calibration curve. Optionally, the dye solution comprises a dye selected from the group consisting of Brilliant-Yellow, Bromocresol-Green, sodium 1,2-naphthoquinone-4-sulfonate, Thymol-Blue, Bromophenol Blue, Clayton-Yellow, and combinations thereof. Optionally, the dye solution further comprises a solvent for the dye. Optionally, the concentration of the dye is in a range of 0.005 to 5 wt % of the solvent. Optionally, the dye solution has a pH in a range of 1 to 5. Optionally, the reference emulsifier and the emulsifier comprise an amidoamine emulsifier. Optionally, the method further comprises determining a targeted testing wavelength of the emulsifier and dye complex in a sample. Optionally, the reference samples further comprise an ingredient selected from the group consisting of water, a water-soluble salt, lime, a weighting agent, a suspension agent, a viscosifier, a dispersant, a friction reducer, a fluid loss additive, a rheology modifier, lubricants, simulated drilled solids, and combinations thereof. Optionally, the ratio of the volume of treatment fluid to the volume of the dye solution is in a range of 3:1 to 1:1. Optionally, the supernatant is separated from the solids via centrifugation, and wherein the centrifugation is performed at a speed in a range of 500 to 4,500 revolutions per minute. Optionally, the centrifugation is performed for a period of time in a range of 1 minute to 120 minutes. Optionally, the absorbance of the test sample is measured at a wellsite or a mud plant using a colorimeter. Optionally, the method further comprises adding additional emulsifier to the treatment fluid after determination of the concentration of emulsifier in the test sample to bring the total concentration of emulsifier in the treatment fluid to within a range of a target concentration. Optionally, the reference samples consist of every ingredient in the treatment fluid and in the same concentrations as the treatment fluid except for the different concentrations of the reference emulsifier. Optionally, the method further comprises obtaining or generating multiple calibration curves from different sets of reference samples, wherein each set of reference samples has a different treatment fluid property than other sets of reference samples. Optionally, the method further comprises: determining a correction factor from control test samples; and applying the correction factor to the determined concentration of residual emulsifier in the test sample. Optionally, the concentration of the emulsifier that is determined is an initial concentration of emulsifier prior to using the treatment fluid in an oil or gas operation. Optionally, the concentration of the emulsifier that is determined is a residual concentration of emulsifier during or after use of the treatment fluid in an oil or gas operation.

Another embodiment of the present disclosure is a method comprising: 1) forming a volume of drilling fluid at a mud plant, wherein the drilling fluid comprises a hydrocarbon liquid and an emulsifier; 2) preparing a test sample comprising the steps of: (i) obtaining a volume of the drilling fluid after the drilling fluid is formed at the mud plant; (ii) adding a volume of a dye solution to the volume of drilling fluid, wherein an oil-soluble emulsifier and dye complex is formed; (iii) separating a supernatant from solids in the drilling fluid, wherein the supernatant comprises the oil-soluble emulsifier and dye complex; and (iv) removing a volume of the separated supernatant, wherein the supernatant is the test sample; and 3) determining a concentration of the emulsifier in the drilling fluid by comparing the test sample against a reference sample, wherein the reference sample comprises: (i) a known concentration of a reference emulsifier; (ii) the dye solution; and (iii) a hydrocarbon liquid. Optionally, the concentration of the emulsifier that is determined is an initial concentration of emulsifier prior to using the drilling fluid in a drilling operation. Optionally, the comparison against the reference sample comprises: a visual comparison of a color of the test sample against a color of the reference sample; or measuring an absorbance of the test sample and the reference sample using an instrument and then comparing the absorbance values of the test sample and reference sample. Optionally, the method further comprises preparing more than one reference sample, wherein each of the reference samples have a different known concentration of the reference emulsifier. Optionally, the method further comprises adding incremental amounts of the emulsifier to another volume of the drilling fluid until the color of the supernatant approximates the color of the reference sample. Optionally, the method further comprises preparing more than one reference sample, wherein each of the reference samples have a different known concentration of the reference emulsifier, and wherein each of the reference samples has a different color from each other; and wherein the determination of the concentration of emulsifier in the drilling fluid comprises visually comparing a color of the test sample to the colors of the reference samples and determining which reference sample the color of the test sample most closely matches. Optionally, the method further comprises measuring an absorbance of the reference sample using an instrument; and measuring an absorbance of the test sample using an instrument, wherein the comparison against the reference sample comprises comparing the absorbance of the test sample against the absorbance of the reference sample. Optionally, the dye solution comprises a dye selected from the group consisting of Brilliant-Yellow, Bromocresol-Green, sodium 1,2-naphthoquinone-4-sulfonate, Thymol-Blue, Bromophenol Blue, Clayton-Yellow, and combinations thereof. Optionally, the dye solution further comprises a solvent for the dye. Optionally, the concentration of the dye is in a range of 0.005 to 5 wt % of the solvent. Optionally, the dye solution has a pH in a range of 1 to 5. Optionally, the reference emulsifier and the emulsifier comprise an amidoamine emulsifier. Optionally, the reference sample further comprises an ingredient selected from the group consisting of water, a water-soluble salt, lime, a weighting agent, a suspension agent, a viscosifier, a dispersant, a friction reducer, a fluid loss additive, a rheology modifier, lubricants, simulated drilled solids, and combinations thereof. Optionally, the ratio of the volume of the drilling fluid to the volume of the dye solution is in a range of 3:1 to 1:1. Optionally, the supernatant is separated from the solids via centrifugation, and wherein the centrifugation is performed at a speed in a range of 500 to 4,500 revolutions per minute. Optionally, the centrifugation is performed for a period of time in a range of 1 minute to 120 minutes. Optionally, the method further comprises adding additional emulsifier to the drilling fluid after determination of the concentration of emulsifier in the test sample to bring the total concentration of emulsifier in the drilling fluid to within a range of a target concentration.

Another embodiment of the present disclosure is a method comprising: 1) introducing a drilling fluid into a subterranean formation for forming a wellbore, wherein the drilling fluid comprises a hydrocarbon liquid and an initial concentration of the emulsifier; 2) preparing a test sample comprising the steps of: (i) obtaining a volume of the drilling fluid during or after formation of the wellbore; (ii) adding a volume of a dye solution to the volume of drilling fluid, wherein an oil-soluble emulsifier and dye complex is formed when a residual concentration of the emulsifier in the drilling fluid is greater than 0 pounds per barrel; (iii) separating a supernatant from solids in the drilling fluid, wherein the supernatant comprises the oil-soluble emulsifier and dye complex, if formed; and (iv) removing a volume of the separated supernatant, wherein the supernatant is the test sample; and 3) determining the residual concentration of the emulsifier in the drilling fluid by comparing the test sample against a reference sample, wherein the reference sample comprises: (i) a known concentration of a reference emulsifier; (ii) the dye solution; and (iii) a hydrocarbon liquid. Optionally, the comparison against the reference sample comprises: a visual comparison of a color of the test sample against a color of the reference sample; or measuring an absorbance of the test sample and the reference sample using an instrument and then comparing the absorbance values of the test sample and reference sample. Optionally, the method further comprises preparing more than one reference sample, wherein each of the reference samples have a different known concentration of the reference emulsifier. Optionally, the method further comprises adding incremental amounts of the emulsifier to another volume of the drilling fluid until the color of the supernatant approximates the color of the reference sample. Optionally, the method further comprises preparing more than one reference sample, wherein each of the reference samples have a different known concentration of the reference emulsifier, and wherein each of the reference samples has a different color from each other; and wherein the determination of the concentration of emulsifier in the drilling fluid comprises visually comparing a color of the test sample to the colors of the reference samples and determining which reference sample the color of the test sample most closely matches. Optionally, the method further comprises measuring an absorbance of the reference sample using an instrument; and measuring an absorbance of the test sample using an instrument, wherein the comparison against the reference sample comprises comparing the absorbance of the test sample against the absorbance of the reference sample. Optionally, the dye solution comprises a dye selected from the group consisting of Brilliant-Yellow, Bromocresol-Green, sodium 1,2-naphthoquinone-4-sulfonate, Thymol-Blue, Bromophenol Blue, Clayton-Yellow, and combinations thereof. Optionally, the dye solution further comprises a solvent for the dye. Optionally, the concentration of the dye is in a range of 0.005 to 5 wt % of the solvent. Optionally, the dye solution has a pH in a range of 1 to 5. Optionally, the reference emulsifier and the emulsifier comprise an amidoamine emulsifier. Optionally, the reference sample further comprises an ingredient selected from the group consisting of water, a water-soluble salt, lime, a weighting agent, a suspension agent, a viscosifier, a dispersant, a friction reducer, a fluid loss additive, a rheology modifier, lubricants, simulated drilled solids, and combinations thereof. Optionally, the ratio of the volume of the drilling fluid to the volume of the dye solution is in a range of 3:1 to 1:1. Optionally, the supernatant is separated from the solids via centrifugation, and wherein the centrifugation is performed at a speed in a range of 500 to 4,500 revolutions per minute. Optionally, the centrifugation is performed for a period of time in a range of 1 minute to 120 minutes. Optionally, the method further comprises adding additional emulsifier to the drilling fluid after determination of the residual concentration of emulsifier in the test sample to bring the total concentration of emulsifier in the drilling fluid to within a range of a target concentration.

### Examples

To facilitate a better understanding of the various embodiments, the following examples are given. In the examples for FIGS. 2A-2E, 3-5, and 10-12 below, the emulsifier was an amidoamine emulsifier and the peak wavelength was pre-determined to be 600 nm. Absorbance was measured on a UV-Vis spectrometer. The dye solution in the examples below included 0.005 to 0.01 wt. % of Bromophenol Blue, ethyl alcohol as the solvent, water, and acetic acid to adjust the pH within a range of 3 to 4. The dye solution was added to the base fluid or drilling fluid at a ratio of 1:2 volume by volume (v/v).

The reference samples that were prepared for FIG. 4 included a cyclic and branched isoparaffinic oil and the dye solution as the base fluid. The graph shows a mostly linear plot of absorbance versus emulsifier concentration ranging from 0 to 9 weight percent of the base fluid with an R² of 0.9953. This shows the oil-soluble emulsifier and dye complex was formed.

The reference samples that were prepared for FIG. 5 were formulated to simulate a drilling fluid. The ingredients and concentrations are listed in Table 1. The drilling fluid reference samples had an oil-to-water ratio (OWR) of 70:30 and a density of 12 pounds per gallon (“ppg”) (1.44 kilograms per liter “kg/L”). The graph shows an almost perfectly linear plot of absorbance versus emulsifier concentration in the drilling fluid reference samples ranging from 0 to 20 ppb (0 to 77.3 kg/m³) and the calibration curve with an R² of 0.9965. This shows that the oil-soluble emulsifier and dye complex formed in a representative drilling fluid.

TABLE 1

| Ingredients | Concentration in ppb (kg/m³) |
|---|---|
| Base oil | To achieve OWR of 70:30 |
| Emulsifier | 0 to 20 (0 to 77.3) |
| Lime | 4 (15.5) |
| Polymeric fluid loss control agent | 2 (7.7) |
| Rheology modifier | 3.25 (12.6) |
| Suspending agent | 13 (50.2) |
| Salt | 32.31 (124.9) |
| Water | 84.54 (386.5) |
| API Barite, Weighting agent | To achieve density of 12 ppg |

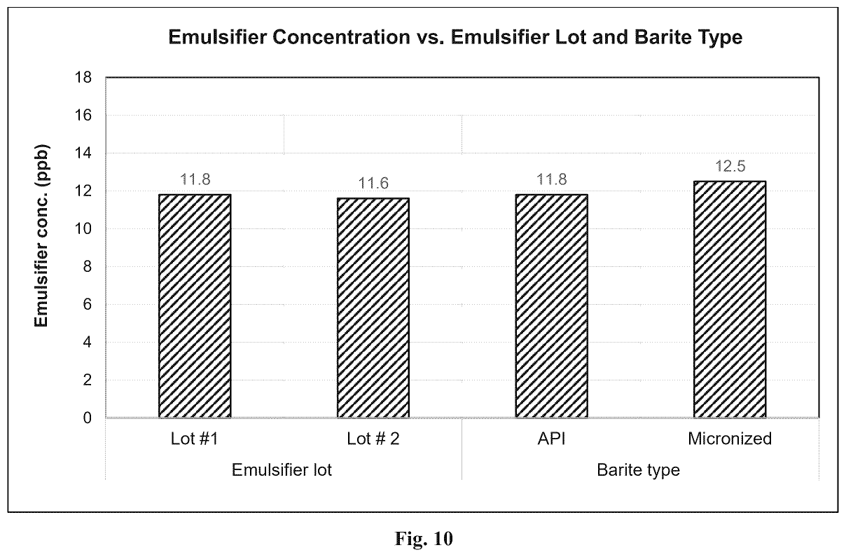

The data presented in the bar graphs for FIGS. 10-12 were to evaluate the effect of different additive concentrations at a known concentration of emulsifier of 12 ppb (46.4 kg/m³) against the calibration curve. That is, each of the test samples contained the emulsifier at the stated concentration and the dye solution with variances in the other additives. The calibration curve that was used to determine concentration of emulsifier was the calibration curve from FIG. 5.

FIG. 10 evaluated different emulsifier manufacturing lots and barite type. As can be seen in FIG. 5, the concentration of the emulsifier reflected a slightly lower concentration than actual for lot #1 and lot #2, however the determined values against the calibration curve were very close to actual (12 ppb). This indicates that different lots from the same emulsifier supplier had little effect on the determined value versus actual. API barite has a particle size of 200 mesh and micron-sized barite had a d50 of less than 5 micrometers. The API barite type was also very close to the actual value; whereas micron-sized barite gave a value that was slightly higher than actual. This indicates that the particle size of a weighting agent may produce determined values against the calibration curve that are higher than actual values.

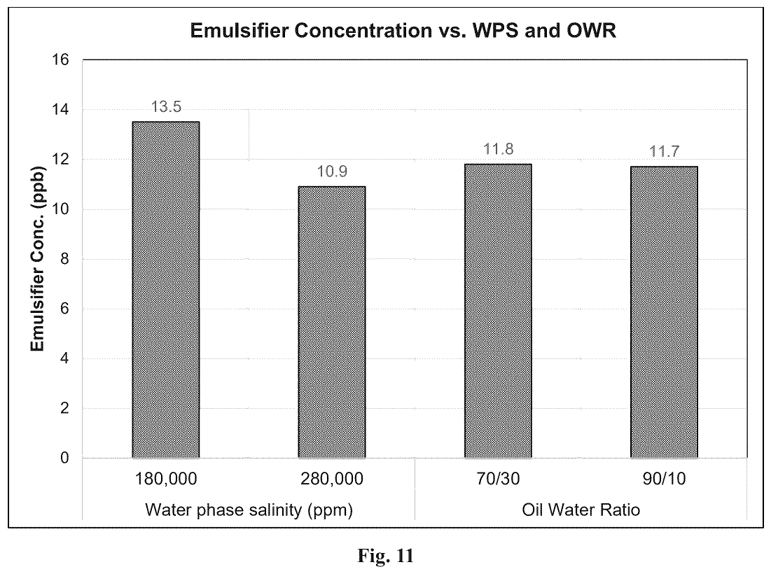

FIG. 11 evaluated different water phase salinities (WPS) and oil-to-water ratios (OWR). As can be seen in FIG. 11, the concentration of the emulsifier reflected a higher concentration than actual when the WPS was 180,000 and a lower concentration when the WPS was 280,000. This indicates that the WPS can play a role in affecting the determined concentration values versus actual. This also indicates that different calibration curves can be generated with different WPS, or a correction factor may need to be used. The OWR had very little effect on the determined values to actual. This indicates that the OWR may not play as big a role as WPS.

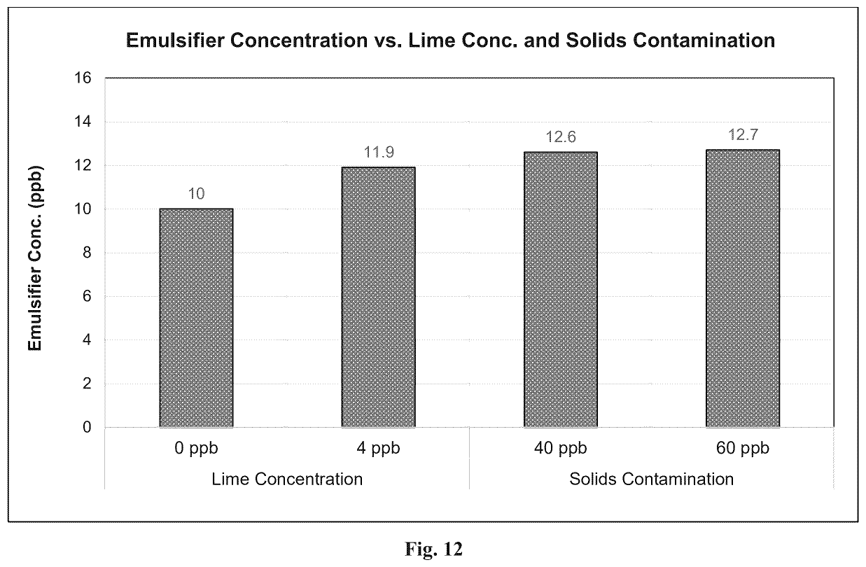

FIG. 12 evaluated different concentrations of lime and solids (weighting agent). As can be seen in FIG. 12, the concentration of the emulsifier decreased by 2 ppb when no lime was present but was extremely close to the actual value at 4 ppb. This indicates that the lime concentration can play a role in affecting the determined concentration values to actual. This also indicates that different calibration curves may need to be generated with each calibration curve having different lime concentrations, or a correction factor may need to be used. The weighting agent concentrations also yielded almost identical values, which were both slightly higher than the actual value. This indicates that the weighting agent concentration may consistently produce determined values against the calibration curve that are higher than actual values, regardless of the concentration of the weighting agent.

Therefore, the various embodiments are well adapted to attain the ends and advantages mentioned as well as those that are inherent therein. The particular embodiments disclosed above are illustrative only, as the various embodiments may be modified and practiced in different but equivalent manners apparent to those skilled in the art having the benefit of the teachings herein. Furthermore, no limitations are intended to the details of construction or design herein shown, other than as described in the claims below. It is, therefore, evident that the particular illustrative embodiments disclosed above may be altered or modified and all such variations are considered within the scope and spirit of the present invention.

As used herein, the words “comprise,” “have,” “include,” and all grammatical variations thereof are each intended to have an open, non-limiting meaning that does not exclude additional elements or steps. While compositions, systems, and methods are described in terms of “comprising,” “containing,” or “including” various components or steps, the compositions, systems, and methods also can “consist essentially of” or “consist of” the various components and steps. It should also be understood that, as used herein, “first,” “second,” and “third,” are assigned arbitrarily and are merely intended to differentiate between two or more fluids, sets of samples, calibration curves etc., as the case may be, and do not indicate any sequence. Furthermore, it is to be understood that the mere use of the word “first” does not require that there be any “second,” and the mere use of the word “second” does not require that there be any “third,” etc.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent(s) or other documents that may be incorporated herein by reference, the definitions that are consistent with this specification should be adopted.

What is claimed is:

1. A method comprising:
1) preparing a test sample comprising the steps of:
(i) obtaining a volume of a treatment fluid;
(ii) adding a volume of a dye solution to the volume of treatment fluid, wherein an oil-soluble emulsifier and dye complex is formed when the concentration of an emulsifier in the treatment fluid is greater than 0 pounds per barrel;
(iii) separating a supernatant from solids in the treatment fluid, wherein the supernatant comprises the oil-soluble emulsifier and dye complex, if formed; and
(iv) removing a volume of the supernatant, wherein the supernatant is the test sample; and
2) determining a concentration of the emulsifier in the treatment fluid by comparing the test sample against a reference sample, wherein the reference sample comprises:
(i) a known concentration of a reference emulsifier;
(ii) the dye solution; and
(iii) a hydrocarbon liquid.

2. The method according to claim 1, wherein the concentration of the emulsifier that is determined is an initial concentration of emulsifier prior to using the treatment fluid in an oil and gas operation.

3. The method according to claim 1, wherein the concentration of the emulsifier that is determined is a residual concentration of emulsifier during or after use of the treatment fluid in an oil or gas operation.

4. The method according to claim 1, wherein the comparison against the reference sample comprises visually comparing a color of the test sample against a color of the reference sample.

5. The method according to claim 4, further comprising adding incremental amounts of the emulsifier to another volume of the treatment fluid until the color of the supernatant approximates the color of the reference sample.

6. The method according to claim 1, further comprising:
preparing more than one reference sample, wherein each of the reference samples have a different known concentration of the reference emulsifier, and wherein each of the reference samples has a different color from each other; and
wherein the determination of the concentration of emulsifier in the treatment fluid comprises visually comparing a color of the test sample to the colors of the reference samples and determining which reference sample the color of the test sample most closely matches.

7. The method according to claim 1, further comprising:
measuring an absorbance of the reference sample using an instrument; and
measuring an absorbance of the test sample using an instrument,
wherein the comparison against the reference sample comprises comparing the absorbance of the test sample against the absorbance of the reference sample.

8. A method comprising:
obtaining or generating a calibration curve correlating absorbance to an emulsifier concentration from reference samples, wherein the reference samples comprise:
different concentrations of a reference emulsifier;
a dye solution; and
a hydrocarbon liquid;
preparing a test sample comprising the steps of:
obtaining a volume of a treatment fluid;
adding a volume of the dye solution to the volume of the treatment fluid, wherein an oil-soluble emulsifier and dye complex is formed when the concentration of an emulsifier in the treatment fluid is greater than 0 pounds per barrel;
separating a supernatant from solids in the treatment fluid, wherein the supernatant comprises the oil-soluble emulsifier and dye complex, if formed; and
removing a volume of the separated supernatant, wherein the volume of the supernatant is the test sample;
measuring an absorbance of the test sample using an instrument; and
determining a concentration of the emulsifier in the test sample by comparing the measured absorbance to the calibration curve.

9. The method according to claim 8, wherein the dye solution comprises a dye selected from the group consisting of Brilliant-Yellow, Bromocresol-Green, sodium 1,2-naphthoquinone-4-sulfonate, Thymol-Blue, Bromophenol Blue, Clayton-Yellow, and combinations thereof.

10. The method according to claim 9, wherein the dye solution further comprises a solvent for the dye.

11. The method according to claim 10, wherein the concentration of the dye is in a range of 0.005 to 5 wt % of the solvent.

12. The method according to claim 8, wherein the dye solution has a pH in a range of 1 to 5.

13. The method according to claim 8, wherein the reference emulsifier and the emulsifier comprise an amidoamine emulsifier.

14. The method according to claim 8, further comprising determining a targeted testing wavelength of the emulsifier and dye complex in a sample.

15. The method according to claim 8, wherein the reference samples further comprise an ingredient selected from the group consisting of water, a water-soluble salt, lime, a weighting agent, a suspension agent, a viscosifier, a dispersant, a friction reducer, a fluid loss additive, a rheology modifier, lubricants, simulated drilled solids, and combinations thereof.

16. The method according to claim 8, wherein the ratio of the volume of treatment fluid to the volume of the dye solution is in a range of 3:1 to 1:1.

17. The method according to claim 8, wherein the supernatant is separated from the solids via centrifugation, and wherein the centrifugation is performed at a speed in a range of 500 to 4,500 revolutions per minute.

18. The method according to claim 17, wherein the centrifugation is performed for a period of time in a range of 1 minute to 120 minutes.

19. The method according to claim 8, wherein the absorbance of the test sample is measured at a wellsite or a mud plant using a colorimeter.

20. The method according to claim 8, further comprising adding additional emulsifier to the treatment fluid after determination of the concentration of emulsifier in the test sample to bring the total concentration of emulsifier in the treatment fluid to within a range of a target concentration.

21. The method according to claim 8, wherein the reference samples consist of every ingredient in the treatment fluid and in the same concentrations as the treatment fluid except for the different concentrations of the reference emulsifier.

22. The method according to claim 8, further comprising obtaining or generating multiple calibration curves from different sets of reference samples, wherein each set of reference samples has a different treatment fluid property than other sets of reference samples.

23. The method according to claim 8, further comprising:
determining a correction factor from control test samples; and
applying the correction factor to the determined concentration of residual emulsifier in the test sample.

24. The method according to claim 8, wherein the concentration of the emulsifier that is determined is an initial concentration of emulsifier prior to using the treatment fluid in an oil or gas operation.

25. The method according to claim 8, wherein the concentration of the emulsifier that is determined is a residual concentration of emulsifier during or after use of the treatment fluid in an oil or gas operation.

26. A method comprising:
1) forming a volume of drilling fluid at a mud plant, wherein the drilling fluid comprises a hydrocarbon liquid and an emulsifier;
2) preparing a test sample comprising the steps of:
(i) obtaining a volume of the drilling fluid after the drilling fluid is formed at the mud plant;
(ii) adding a volume of a dye solution to the volume of drilling fluid, wherein an oil-soluble emulsifier and dye complex is formed;
(iii) separating a supernatant from solids in the drilling fluid, wherein the supernatant comprises the oil-soluble emulsifier and dye complex; and
(iv) removing a volume of the separated supernatant, wherein the supernatant is the test sample; and
3) determining a concentration of the emulsifier in the drilling fluid by comparing the test sample against a reference sample, wherein the reference sample comprises:
(i) a known concentration of a reference emulsifier;
(ii) the dye solution; and
(iii) a hydrocarbon liquid.

27. The method according to claim 26, wherein the concentration of the emulsifier that is determined is an initial concentration of emulsifier prior to using the drilling fluid in a drilling operation.

28. The method according to claim 26, wherein the comparison against the reference sample comprises: a visual comparison of a color of the test sample against a color of the reference sample; or measuring an absorbance of the test sample and the reference sample using an instrument and then comparing the absorbance values of the test sample and reference sample.

29. The method according to claim 26, further comprising preparing more than one reference sample, wherein each of the reference samples have a different known concentration of the reference emulsifier.

30. A method comprising:
1) introducing a drilling fluid into a subterranean formation for forming a wellbore, wherein the drilling fluid comprises a hydrocarbon liquid and an initial concentration of the emulsifier;
2) preparing a test sample comprising the steps of:
(i) obtaining a volume of the drilling fluid during or after formation of the wellbore;
(ii) adding a volume of a dye solution to the volume of drilling fluid, wherein an oil-soluble emulsifier and dye complex is formed when a residual concentration of the emulsifier in the drilling fluid is greater than 0 pounds per barrel;
(iii) separating a supernatant from solids in the drilling fluid, wherein the supernatant comprises the oil-soluble emulsifier and dye complex, if formed; and
(iv) removing a volume of the separated supernatant, wherein the supernatant is the test sample; and
3) determining the residual concentration of the emulsifier in the drilling fluid by comparing the test sample against a reference sample, wherein the reference sample comprises:
(i) a known concentration of a reference emulsifier;
(ii) the dye solution; and
(iii) a hydrocarbon liquid.

31. The method according to claim 30, wherein the comparison against the reference sample comprises: a visual comparison of a color of the test sample against a color of the reference sample; or measuring an absorbance of the test sample and the reference sample using an instrument and then comparing the absorbance values.

32. The method according to claim 30, further comprising preparing more than one reference sample, wherein each of the reference samples have a different known concentration of the reference emulsifier.
